SEQUENCE LISTING (10 sequences)

SEQ ID NO: 1 - DNA, 18 bp, Homo sapiens
gaaggtgaaggtcggagt

SEQ ID NO: 2 - DNA, 20 bp, Homo sapiens
gaagatggtgatgggatttc

SEQ ID NO: 3 - DNA, 20 bp, Homo sapiens
aatcgagaggcacattacgg

SEQ ID NO: 4 - DNA, 20 bp, Homo sapiens
gttgaaaatggcccagaaga

SEQ ID NO: 5 - DNA, 20 bp, Homo sapiens
ttgtcttcctgctcatggtg

SEQ ID NO: 6 - DNA, 20 bp, Homo sapiens
tcagcatctcggcaagagta

SEQ ID NO: 7 - DNA, 20 bp, Homo sapiens
tgctcattttgcttgtctgg

SEQ ID NO: 8 - DNA, 20 bp, Homo sapiens
gccatacatgtcctcgtcct

SEQ ID NO: 9 - DNA, 20 bp, Homo sapiens
cttcgcaagcctgctactct

SEQ ID NO: 10 - DNA, 20 bp, Homo sapiens
ggctttgtggtcaaaggtgt

(19) United States
(12) Patent Application Publication
(10) Pub. No.: US 2013/0045947 A1
(43) Pub. Date: Feb. 21, 2013

(54) PLA2ACTIVITY AS A MARKER FOR OVARIAN AND OTHER GYNECOLOGIC CANCERS

(71) Applicant: Yan Xu, Indianapolis, IN (US)

(72) Inventor: Yan Xu, Indianapolis, IN (US)

(73) Assignee: INDIANA UNIVERSITY RESEARCH AND TECHNOLOGY CORPORATION, Indianapolis, IN (US)

(21) Appl. No.: 13/584,667

(22) Filed: Aug. 13, 2012

Related U.S. Application Data
(63) Continuation-in-part of application No. PCT/US2011/024658, filed on Feb. 11, 2011.
(60) Provisional application No. 61/303,509, filed on Feb. 11, 2010.
(60) Provisional application No. 61/412,364, filed on Nov. 10, 2010.

(51) Int. Cl. C12Q 1/44; C12Q 1/02; A61P 35/00; A61K 31/724; A61K 31/366; G01N 21/75; A61K 31/4965

(52) U.S. Cl. 514/58; 435/19; 435/29; 514/255.06; 514/460

(57) ABSTRACT

Materials and Methods are provided for the diagnosis, monitoring, and personalized treatments of gynecological cancers. The methods comprise determining levels of PLA₂ activity in sample of tissue or fluid recovered from patient; elevated levels of PLA₂ activity are consistent with epithelial ovarian cancer (EOC). These methods include assaying for PLA₂ activity within tissue, ascites, blood, and other tissue forms by exposing the patient sample to a fluorogenic compound such as DBPC. The methods disclosed herein further include correlating the fluorogenic detection with a disease state in the patient, including diseases such as gynecological cancers, such as EOC. The methods comprise determining levels of total PLA₂ activity, and of specific isoforms of PLA₂ such as iPLA₂, iPLA₂β, cPLA₂, among other isoforms.

### PRIORITY CLAIM

This application is a continuation-in-part of International Patent Application Number PCT/US2011/024658, which has an International Filing Date of Feb. 11, 2011, and which itself claims the benefit of U.S. Provisional Patent Application Nos. 61/303,509 filed on Feb. 11, 2010, and 61/412,364 filed on Nov. 10, 2010; each of these patent applications is incorporated herein by reference in its entirety as if each were incorporated herein by reference individually.

### FIELD OF THE DISCLOSURE

The present disclosure relates generally to cancer. More particularly, the present disclosure relates to phospholipase A₂ (PLA₂) as a novel marker and as a target for diagnosing, evaluating and treating ovarian and other gynecologic cancers.

### BACKGROUND

Gynecological cancers, and specifically ovarian, uterine, and cervical cancers, cause more than 26,000 deaths annually in the United States. A majority of epithelial ovarian cancer (EOC) patients present with late-stage disease. Curative treatment for late-stage EOC, and in particular for refractory and drug-resistant EOC, is often ineffective resulting in high mortality rates from EOC.

In general, there is not an acceptable method for screening patients for EOC. Current screening methods, such as ultrasound, are inadequate for early stage detection and in some cases for staging of EOC patient. Further, current markers for EOC, in general, are poor distinguishers for distinguishing between benign and malignant tumor development. Hence, development of markers for monitoring disease progress which also function as better targets for personalized treatment is urgently needed for a majority of EOC patients.

Therefore, a method and marker for better diagnosis, monitoring of gynecological cancer progression, and application in personalized gynecological cancer treatment are desirable. Some aspects of the present disclosure, disclosed herein, address these needs.

### SUMMARY OF THE DISCLOSURE

Some embodiments of the disclosure include methods for evaluating a disease such a EOC including detecting and/or following the course of a disease including, for example, diagnosing, prognosing and evaluating the efficacy of treating a given disease, these methods may include the steps of measuring PLA₂ activity in a patient sample and providing information associated with correlating the PLA₂ activity measured in said step of measuring, with a PLA₂ activity value indicative of a disease. Some embodiments further include the step of comparing the values measured in a given sample with valued indicative of various diseases and concluding that a given sample includes evidence of disease. Some embodiments of the methods disclosed herein include measuring the activity of PLA₂ comprising, for example, iPLA₂. Some further embodiments include methods of correlating a PLA₂ activity value with a PLA₂ activity value indicative of a form of gynecological cancer. In further embodiments, the form of gynecological cancer is EOC.

Some embodiments further include the step of obtaining the patient sample from a patient. In some embodiments of the disclosure, the patient sample may be selected form the group of sample consisting of tissue samples, cells, blood, bodily fluids, cellular fluids, discharges or fluids within or otherwise associated or produced by tumors, cysts or other growths and the like.

In some further embodiments of the methods disclosed herein, the step of measuring PLA₂ activity in a patient sample involves using a fluorogenic compound, including but not limited to the fluorogenic compound DBPC. Still other embodiments include the use of still other compounds that produce a detectable signal and can be used to measure or at least estimate PLA₂ activity in a given sample. In still other embodiments of the disclosed methods, the measuring step involves the use of radioactive compounds. Some embodiments may include further separation steps and/or sample preparations to increase the sensitivity and or reproduce-ability of the activity assay. Still another embodiment includes normalizing the level of PLA₂ activity measured in a given to sample to the number of cells associated with the cells or to the level of protein in the sample.

In even further embodiments of the methods disclosed herein, the step of providing information comprises providing information correlating PLA₂ activity in range of activity that is demonstratively higher in sample for a patent with a form of GYN cancer such as EOC. In other embodiments of the methods disclosed herein, the step of providing information comprises providing information correlating PLA₂ activity in the range of about 1.5 higher than that detected in similar but health tissue of the same type. In still other embodiments, the level of activity that correlates with disease may be about 2× or higher than normal activity measured in sample form non-cancerous sources include healthy samples or at least benign samples.

Still other embodiments of the methods provided herein, include the step of introducing a compound to the patient which inhibits activity of at least one isoform of PLA₂. In some embodiments, the isoforms of PLA₂ in the sample includes at least one of the following sPLA₂, cPLA₂ and iPLA₂. Inhibitors that can be used include, but are not limited to, BEL. Some embodiments may include the steps of conducting a given PLA₂ assay with and without the presence of a given PLA₂ inhibitor and using any difference observed in the assays to assign at least a portion of the reduced activity to the forms of PLA₂ thought to be sensitive to the specific inhibitor used in the assay.

Further embodiments of the methods described herein includes a method of detecting PLA₂ activity within a sample comprising the steps of introducing a fluorogenic compound to a sample, and detecting a signal produced by the fluorogenic compound in the presence of the sample. In some embodiments of the method described herein the sample is a tissue and the method further includes the step of fixing the tissue.

In yet further embodiments of the methods disclosed herein, the tissue sample is homogenized and the fluorogenic compound is introduced to the supernant resulting from the homogenization of the tissue sample.

Still further embodiments of the methods include the step of correlating the signal value, detected in the step of detecting, with a disease. Some embodiments of these methods include the disease being a form of cancer. In some embodiments, the form of cancer is EOC.

Some embodiments include identifying a marker for diseases such as, but not necessarily limited to, EOC. These markers include phospholipase A₂ (PLA₂) activity that is demonstratively higher in samples such as fluids, tissues, blood, cells and like associated with disease such as EOC than it is in samples of healthy sources or samples from benign tumors.

In some embodiments, demonstratively higher activity indicative of a disease state is at least 1.5 or in some embodiments at least 2 fold higher in a sample from a patient with a disease then it is in a patient that is without disease or that presents with a benign tumor.

In some embodiments the identification of the maker is indicative of cancer, especially GYN cancers such as EOC. In some embodiments the marker can be used to diagnose a disease, prognosticate on the course of the disease, following the course of a disease or to evaluate the effectiveness of a treatment of a disease. In some embodiment the maker is activity that due primarily to elevated iPLA₂β activity. In some embodiments values of PLA₂ activity used as markers for disease are used to personalize the treatment of a patient.

Some of the components of Ascites associated with epithelial ovarian cancer (EOC) promote tumor development by mechanisms that are incompletely understood. Lysophosphatidic acid (LPA), a major tumor-promoting factor in EOC ascites, is an enzymatic product of the enzymes autotaxin (ATX) and phospholipase A₂ (PLA₂). The contribution of PLA₂ activities to ovarian tumorigenesis was investigated. The quantitative measurement of PLA₂ activities in ascites and tissues as well as assay conditions selective for PLA₂ subtypes were optimized and validated. PLA₂ activities correlated with tumor-promoting activates in cell-based and in vivo assays. High activities consistent with both cytosolic and calcium-independent PLA₂ were found in human EOC ascites for the first time. Elevated PLA₂ and ATX activities were also observed in EOC compared to benign tumors and normal tissues. Cell-free and vesicle-free (S4) human EOC ascites potently promoted proliferation, migration, and invasion of human EOC cells in a PLA₂-dependent manner. LPA appears to mediate a significant part of the cell-stimulating effects of ascites. The S4 ascites fraction stimulated tumorigenesis/metastasis in vivo. The compound methyl arachidonvi fluorophosphonate was highly effective in inhibiting EOC metastasis in mouse xenograft models for the disease. PLA₂ activity was found in conditioned media from both EOC cells and macrophages. Collectively, these results demonstrate that PLA₂ activity is a marker and a potential therapeutic target for EOC.

These and other features, aspects, and advantages of the present invention will become better understood with reference to the following drawings, descriptions and claims.

### SEQUENCE LISTING

SEQ ID NO. 1, Primer sequence for Quantitative real-time PCR of GAPDH-F: 5′-GAAGGTGAAGGTCGGAGT-3′.

SEQ ID NO. 2, Primer sequence for Quantitative real-time PCR of GAPDH-R: 5′-GAAGATGGTGATGGGATTTC-3′.

SEQ ID NO. 3, Primer sequence for Quantitative real-time PCR of LPA₁-F: 5′-AATCGAGAGGCACATTACGG-3′.

SEQ ID NO. 4, Primer sequence for Quantitative real-time PCR of LPA₁-R: 5′-GTTGAAAATGGCCCAGAAGA-3′.

SEQ ID NO. 5, Primer sequence for Quantitative real-time PCR of LPA₂-F: 5′-TTGTCTTCCTGCTCATGGTG-3′.

SEQ ID NO. 6, Primer sequence for Quantitative real-time PCR of LPA₂-R: 5′-TCAGCATCTCGGCAAGAGTA-3′.

SEQ ID NO. 7, Primer sequence for Quantitative real-time PCR of LPA₃-F: 5′-TGCTCATTTTGCTTGTCTGG-3′.

SEQ ID NO. 8, Primer sequence for Quantitative real-time PCR of LPA₃-R: 5′-GCCATACATGTCCTCGTCCT-3′.

SEQ ID NO. 9, Primer sequence for Quantitative real-time PCR of LPA₄-F: 5′-CTTCGCAAGCCTGCTACTCT-3′.

SEQ ID NO. 10, Primer sequence for Quantitative real-time PCR of LPA₄-R: 5′-GGCTTTGTGGTCAAAGGTGT-3′.

### BRIEF DESCRIPTION OF THE FIGURES

The above-mentioned aspects of the present disclosure and the manner of obtaining them will become more apparent, and aspects thereof will be better understood by reference to the following description of the embodiments of the disclosure, taken in conjunction with the accompanying drawings, figures, schemes, graphs, charts, and the like, wherein:

FIG. 1A. Photomicrographs of para-nuclear PLA₂ activity in cells from cancerous tissue.

FIG. 1B. Photomicrographs of para-nuclear PLA₂ activity only in the surface cells in benign tumors.

FIG. 1C. Photomicrographs of PLA₂ activity in the stromal portions of from normal tissue (non-cellular).

FIG. 1D. Photomicrographs of cells treated with an inhibitor of cPLA₂ and/or iPLA₂ (panels b, d) and an inhibitor of sPLA₂ (panel c).

FIG. 1E. Dot plots (panel a) and a histograph (panel b) of fluorescent intensity normalized to cell count.

FIG. 2A. Graph of arachidonic acid (AA) concentrations in measured in non-malignant (N; n=9) and EOC (n=8) ascites samples.

FIG. 2B. Histographs of fluorescent intensity (PLA₂ activity) measured in the blood sample from subjects: healthy controls, patients with benign gynecologic diseases, and patients with ovarian cancer.

FIG. 2C. Graph of PLA₂ activities measured in the cell-free ascites samples mentioned in FIG. 2A.

FIG. 2D. Histographs showing PLA₂ activities in the exosomes and the supernatant (super) portions of ascites samples.

FIG. 3A. Photomicrographs showing PLA₂ activities in mouse tissues measured in mice that were injected with A2780 human EOC cells and PLA₂ activities were analyzed in tumors: a. no DBPC; b. with DBPC; c. DAPI; d. merged.

FIG. 3B. Photomicrographs showing PLA₂ activities from tumors obtained from control (a) and BEL-treated (b) mice. The tumors in BEL-treated mice were significantly smaller and the PLA₂ activities were reduced.

FIG. 3C. Photomicrographs showing PLA₂ activities detected in tumors derived from human SKOV3 EOC cells, and mouse ID-8 cells (D) EOC cells.

FIG. 3D. Photomicrographs showing PLA₂ activities detected in tumors derived from mouse ID-8 cells EOC cells.

FIG. 3E. Photomicrographs of mouse kidney tissue; the numbers in the figures are the quantified PLA₂ activity/cell values. Normal mouse kidney tissues mainly had sPLA₂-like activity, which was not associated with cell nucleus and minimally affected by BEL.

FIG. 4A. Graph of a time dependent curve demonstrating fluorescent intensity has a linear relation with time

FIG. 4B. Graph of a time dependent curve demonstrating fluorescent intensity has linear relation with time

FIG. 4C. Graph of a standard curve demonstrating that fluorescent intensity has a linear relation with DBPC substrate concentration.

FIG. 5A. A graph of PLA₂ activity, created using the radiometric method of measuring and for PLA₂ activity assay; enzymatic activity was measured in 6 tissue homogenates (3 from ovarian tumors (O1-O3) and 3 from benign gynecological tumors (B1-B3)).

FIG. 5B. A representative graph of PLA₂ activity, created using the fluorometric method of measuring and for PLA₂ activity assay using the same tissue homogenates used in FIG. 5A.

FIG. 5C. A graph illustrating the relationship between PLA₂ activities measured using the standard radiometric method (FIG. 5A) and those measured using the DBPC-based fluorometric method (FIG. 5B.); results from the two methods were highly correlative (R²=0.918, P=0.0026).

FIG. 6A. Dot plots showing PLA₂ and ATX activity in units of fluorescence intensity min⁻¹ mg⁻¹ measured in ascites and tissue homogenates. PLA₂ and ATX activities in ascites fractions. Ascites fractions (S1, S3, S4, P2, and P3, n=10 for each group) were analyzed for PLA₂ and ATX activities as described in Materials and Methods. Mean and SD from 2-4 analyses are shown.

FIG. 6B. Bar graph of PLA₂ activity in tissue homogenates (n=10 in each group) measured in normal, benign, and ovarian cancer tissue.

FIG. 6C. Bar graph of PLA₂ activity measured in tumor homogenates, subgroup determination using specific inhibitors, assays for natural PLA₂, i PLA₂, c PLA₂, and s PLA₂.

FIG. 6D. Bar graph of ATX activity measured in tissue homogenates (n=10 in each group). *P<0.05; ***P<0.001 activity measured in control group compared to activity measured in tissue from a patient with Ovarian Cancer.

FIG. 7A. Photomicrographs showing PLA₂ activity and expression in normal ovaries (top), benign tumors (middle), and EOC tumors (bottom). PLA₂ activity was measured: without DBPC (negative control), far left: with DBPC, center left; staining of nucleus DAPI, center right; and merged images, far right.

FIG. 7B. Photomicrographs of tumor PLA₂ activity measured in: negative control (far left); with DBPC (center left); and in the presence of the inhibitors TAPC (250 μM) (center right) and MAFP (40 μM) (far right).

FIG. 8A. Bar graph of absorbance at (OD 555 nm) measured with SKOV3 cells that were treated with the S1 fraction from ascites collected from different patients.

FIG. 8B. Bar graph of absorbance at (OD 555 nm) measured with HEY cells that were treated with the S1 fraction from ascites collected from different patients.

FIG. 8C. The effect of fractions of 3 ascites collected from 3 patients on SKOV3 proliferation measured in terms of OD at 555 nm, *P<0.05.

FIG. 8D. The effect of fractions of 3 ascites collected from 3 patients on HEY cell proliferation measured in terms of OD at 555 nm, **P<0.01;

FIG. 8E. The effect of fractions of 3 ascites collected from 3 patients on CaOV3 cell proliferation measured in terms of OD at 555 nm, ***P<0.001.

FIG. 9A Bar graph of Migratred cells per field measured for SKOV3 cells exposed to Serum Free (SF) and fractions S1, S3, or P3 collected from the ascetis of 10 different Human patient diagnosed with EOC.

FIG. 9B. Bar graph of Migratred cells per field measured for HEY cells (top panel) and CaOV3 cells (bottom panel) exposed to Serum Free (SF) and fractions S1, S3, or P3 collected from the ascetis of 2 different Human patients diagnosed with EOC.

FIG. 9C. Bar graph of Migratred cells per filed measured for SKOV3-VLB cells exposed to Serum Free (SF) and fractions S1, S3, or P3 collected from the ascetis of a Human patient diagnosed with EOC.

FIG. 9D. Bar graph of Invaded cells per filed measured for SKOV3 cells exposed to Serum Free (SF) and fractions S1, S3, or P3 collected from the ascetis of 4 different Human patients diagnosed with EOC.

FIG. 9E. Bar graph of Migratred cells per filed measured for SKOV3-VLB cells exposed to Serum Free (SF) and the S4 fraction collected from the ascetis of 3 Human patients diagnosed with EOC. Effect of ascites fraction S4 on SKOV3 cell migration. *P<0.05; **P<0.01; ***P<0.001 compared to SF medium. Data are mean and SD of at least 3 experiments.

FIG. 10A. Bar graphs of Migrated cells per field measured using SKOV3 cells exposed to SF, Control, or fraction S1 from ascetis of humans (designated as A4 in the left panel, and A10 in the right panel) diagnosed with EOC.

FIG. 10B. Bar graph of the effect of heat treated ascites fractions or pretreatment of SKOV3 cells with Ki16425 (10 μM) on EOC cell migration.

FIG. 10C. Bar graph of the effect of siRNA-mediated knockdown of LPA₁₋₄ in SKOV3 cells on S1-induced cell migration.

FIG. 10D. Bar graph of the effect of LPI, oleic acid, or lyso-PAF on SKOV3 cell migration.

FIG. 10E. Bar graph of normalized LPA receptor mRNA levels from siRNA-treated SKOV3 cells determined by quantitative real-time PCR. Data are mean and SD of three experiments.

FIG. 11A. Photomicrographs: upper panel, photographs of tumors in the peritoneal cavity; the peritoneal wall; and the diaphragm. Tumors are indicated by arrows. Panel lower left: volume of ascites development in each group; panel lower right, total tumors developed in each group by size (n=8 per group). ***P<0.001.

FIG. 11B. Photomicrographs of hematoxylin- and eosin-stained sections of various organs that were collected from representative mice in each treatment group.

FIG. 12A. Bar graph of the % of PLA₂ activity remaining after exposure to EDTA, MAFP, BEL, TAPC, LY311747, pH 4.5 or pH 7.8

FIG. 12B. Bar graph of % activity the control measured for the untreated (control) and upon exposure to 3 different concentrations of MK517, CsA, or glyburide.

FIG. 12C. Bar graph of Migrated cells per field measured in control and upon exposure to CM, MK571, CSA, GLY or MAFP.

### DETAILED DESCRIPTION OF THE DISCLOSURE

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and what is claimed.

As used herein, unless explicitly stated otherwise or clearly implied otherwise, the term ‘about’ refers to a range of values plus or minus 10 percent, e.g., about 1.0 encompasses values from 0.9 to 1.1.

As used herein, unless explicitly stated otherwise or clearly implied otherwise the terms ‘therapeutically effective dose,’ ‘therapeutically effective amounts,’ and the like, refers to a portion of a compound that has a net positive effect on the health and well being of a human or other animal. Therapeutic effects may include an improvement in longevity, quality of life and the like. These effects also may also include a reduced susceptibility to developing disease or deteriorating health or well being. The effects may be immediate realized after a single dose and/or treatment or they may be cumulative realized after a series of doses and/or treatments.

Unless explicitly or implicitly stated otherwise the term, ‘demonstratively’ as used to refer to a property that is integral to given sample and that can be reproduced with sufficient precision and accuracy to show or illustrate the similarity or difference between at least two different portions, samples and the like.

The majority of epithelial ovarian cancer (EOC) patients present with late stage metastatic disease, often accompanied by significant peritoneal ascites, for which there is no curative treatment. Hence, there is an urgent need for improved detection and new targets for more effective modalities to treat late stage EOC. LPA was originally identified as a major tumor-promoting factor in ovarian cancer ascites, showed its effects in EOC, extensive work has been conducted on LPA both in vitro and in vivo. Over-expression or down-regulation of LPA receptors 1-3 (LPA₁₋₃) in several human EOC cell lines and in vivo mouse studies further demonstrated that LPA is involved in EOC development.

LPA is produced by the action of ATX, PLA_{T}, and PLA₂. More than 30 enzymes that possess PLA₂ or related activity have been identified in mammals and they are divided into four groups based on their cellular localization, substrate specificity, and calcium-dependence, including cytosolic (cPLA₂), calcium-independent (iPLA₂), secreted PLA₂ (sPLA₂), and lipoprotein-associated PLA₂ (Lp-PLA₂). sPLA₂ and Lp-PLA₂ are secreted enzymes. In contrast, both cPLA₂ and iPLA₂ are cytosolic enzymes and their cell-free presentation has only been shown to be related to exosomes from RBL-2H3 cells (a mast and basophil cell line). Exosomes are 40-100 nm diameter membrane vesicles released from multivesicular bodies by intact cells that participate in intercellular signaling.

Only in recent years have PLA₂s emerged as cancer targets and most if not all of these studies focus on sPLA₂ and cPLA₂. Aberrant expression of various PLA₂s has been shown in more than 10 different cancer types including breast, lung, and prostate cancers. However, up-regulation of PLA₂s in EOC has not been clearly detected in any of several studies. On the other hand, it has been shown that iPLA₂β is functionally involved in promoting EOC development in vitro and in vivo.

Phospholipase A₂s (PLA₂s) are enzymes commonly found in mammalian tissue. PLA₂s include several protein families, including secreted phospholipase A₂ (sPLA₂), cytosolic phospholipase A₂ (cPLA₂), and Ca²⁺ independent PLA₂ (iPLA₂). All PLA₂s catalyze hydrolysis of phospholipid substrates to generate a lysophospholipid (LPL) and a free fatty acid (FA), e.g., arachidonic acid (AA). AA can be oxygenated to a variety of bioactive eicosanoids. Phosphatidyalcholine (PC) is an abundant lipid substrate in cell membranes and lysophosphatidylcholin (LPC) is a more common product of PLA₂s. LPC is convertible to Lysophosphatidic acid (LPA) by autotaxin (ATX). Additionally, certain PLA₂s, including sPLA₂ and calcium independent iPLA₂ isoforms can utilize phosphatidic acid (PA) as a substrate for directly producing LPA. The compounds, LPA and AA are shown to be important lipid mediators in chemotaxis and/or chemokinesis.

Aberrant expression of some sPLA₂s and cPLA₂ has been shown in some cancer types (not inclusive of EOC). To date, only the sPLA₂ isoform, sPLA₂-XII, has shown potential as a possible predictive indicator of patient outcomes. Heretofore, comparison data between malignant and benign/normal tissues appears to be nonexistent.

In regard to EOC, specifically, mRNA expression levels of 19 different PLA₂ isoforms (sPLA₂ and cPLA₂s) have demonstrated decreased or unchanged transcriptional levels in EOC versus non-tumoral immortalized (EONT) cells.

iPLA₂s, calcium-independent phospholipase A₂ beta, the Group VIA phospholipase A₂, are intracellular enzymes that do not require Ca²⁺ for their catalytic activity. iPLA₂s are, in general, known to be active in phospholipid remodeling, signal transduction, and cell proliferation and apoptosis.

Various iPLA₂ isoforms use PA as a substrate and produce LPA. LPA and AA are shown to be important lipid mediators in chemotaxis and/or chemokinesis. Further, iPLA₂ activity is known to regulate LPA and other lipid concentrations in tumor microenvironment.

Lysophosphatidic acid (LPA) is a bioactive lipid with multiple functions. Epidemiological, animal, and cell culture studies indicate that LPA plays a role in EOC development. Over-expression or down-regulation of LPA receptors 1, 2, and 3 in several human EOC cell lines and in vivo studies, indicate that LPA receptors are involved in EOC development.

LPA activity is suppressible in various manners including inhibiting its production, increasing its degradation or conversion, preventing receptor occupancy, or and interfering with distal signaling pathways.

LPA is producible by the action of lysophospholipase(s) D (lyso-PLD), e.g., autotaxin (ATX), or via phospholipase(s) A₂ (PLA₂), as discussed above.

The activation of iPLA₂β in non-apoptotic EOC cells occurs via a laminin-β₁-integrin-caspase 3 pathway resulting in LPA and AA increase from EOC cells. Also, iPLA₂ activity increases (in association with EOC) as disclosed herein, are not reflective of increased iPLA₂ expression levels in EOC and/or other GYN cancer tissues.

As detailed above, resulting LPA acts as an “oncolipid” in EOC. Unexpectedly, as disclosed herein the activity of PLA₂ in EOC tissues is increased compared to its activity in benign disease and/or normal tissues without a correlated increase in PLA₂ expression levels. The activity of the PLA₂ enzymes (there are over 20 isoforms of phospholipase A₂ enzymes) taught and described herein, are disclosed as utilizable in the assay and methods disclosed herein as useful markers for diagnosing, prognosis, and monitoring EOC progression, as well as a target for personalized therapeutics in treating EOC.

Since the protein activities are directly related to the biological effects, the work reported herein focused on activities rather than on the expression of PLA₂s. It includes examining PLA₂ activities in human EOC tissues, including ascites specimens. The activities from different subgroups of PLA₂s were distinguished using selective inhibitors and/or other reagents. The quantitative fluorescent PLA₂ assays were optimized and validated using human samples. Bioactive lipid concentrations in cell-free ascites samples and different fractions of ascitic samples were measured. The cell-stimulating activities of human EOC ascites samples were tested in cell-based assays and the mechanisms involved were investigated. Moreover, the efficacy of an iPLA₂ and cPLA₂ dual inhibitor was examined in a mouse xenograft model.

As disclosed in the Examples section provided herein, PLA₂ activities have been assessed in cancer vs. normal tissues and the differences in activity between these types of tissues are disclosed herein as usable and valuable in providing an assay for cancer diagnosis and monitoring. Similarly, PLA₂ activities have been assessed in EOC vs. tissue from benign tumors and usable differences between PLA₂ activities measured between these different tissues have been demonstrated to exit.

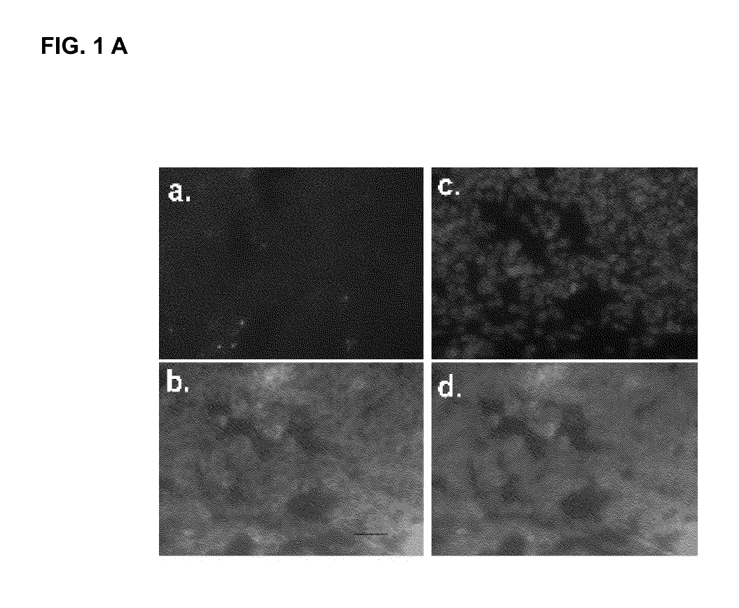

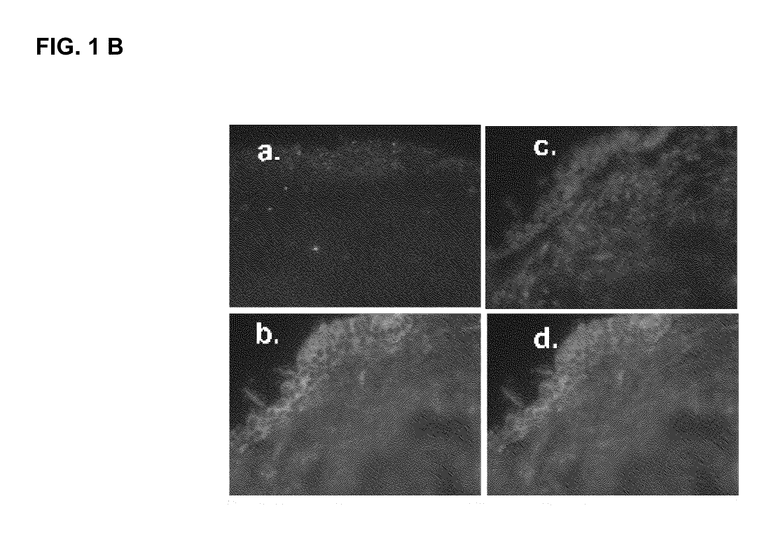

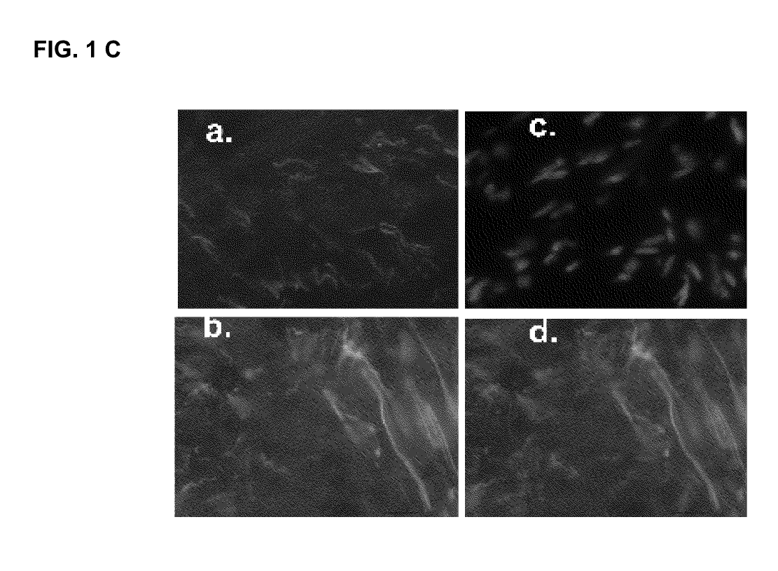

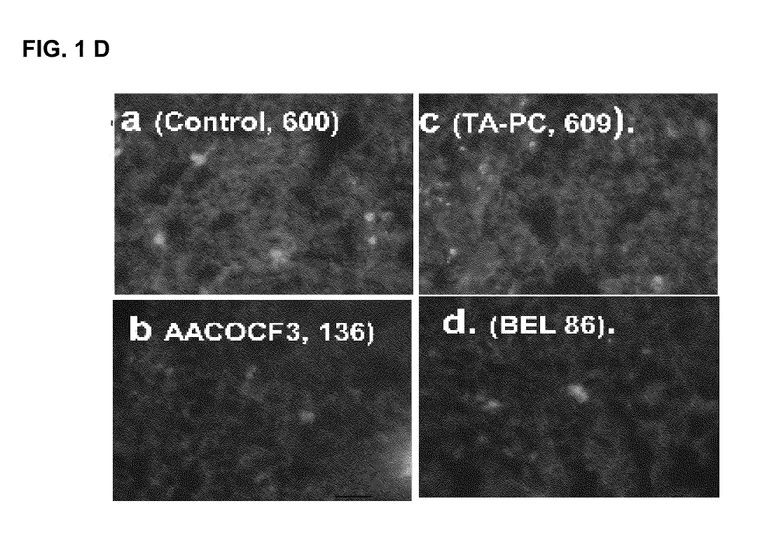

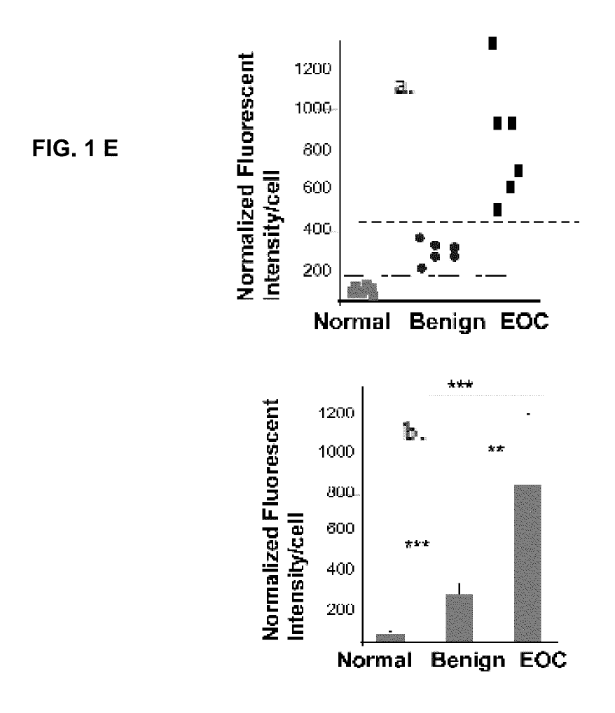

Further, the disclosure provided herein discloses that PLA₂ activity as a robust and useful marker that can be used to distinguishing malignant EOC, and other GYN cancer tissues including, from benign and/or normal tissues. Data in FIG. 1E, shows that PLA₂ activity measured in malignant tissue differs from PLA₂ activity measured in benign tissues. Current markers utilized for EOC (including Ca¹²⁵) lack sufficient differentiation capabilities for distinguishing between benign and malignant tumor development. This common issue suggests that the current markers' biological functions are similar, resulting in difficulty in distinguishing between benign and malignant tumor development. The methods and assays disclosed and described herein provide a further advantage over current markers by providing an assay for distinguishing malignant EOC, and other GYN cancer tissues, from benign and/or normal tissues based on PLA₂ activity.

As disclosed and described herein, PLA₂ activity provides a valuable marker with utility in personalized chemotherapy. As is noted herein, this disclosure provides a method corresponding to greater than 30 percent of all EOC patients (no other genetic changes, other than p53 mutations, is known to occur with an incidence of greater than 30% in EOC patients). Thus, personalized and targeted therapy is the promising goal for EOC treatment.

Further, PLA₂s are involved in cellular processes, sometimes associated in other forms of cancers. The major effects of iPLA₂, for example, in EOC cells are cell migration and invasion, two key steps of tumor metastasis, with a relatively less prominent effect on cell proliferation. Thus, the present embodiments further provides a clinical value (in utilizing of PLA₂ activity as disclosed herein) in cancer types of than EOC. For example, the instant disclosure for analysis of PLA₂ activity as a marker for metastases has a prognostic value in both EOC and other forms of GYN cancers. Hence, it is within the present disclosure to utilize the assay involving PLA₂ activity, disclosed herein, for distinguishing between metastases and primary EOC.

Metastasis is the major cause of death in patients with solid cancer. As such, the assay disclosed herein provides a valuable marker and prognosis tool. Further, the methods associated with PLA₂ activity, disclosed and described herein, provide a valuable and necessary marker adapted for early detection in the diagnosis of GYN diseases.

One example demonstrating the value and novelty of the disclosed embodiments herein is demonstrated in early detection of Type II EOC. Recent data indicates that there exists two major types of EOC, Type I and Type II. Type I tumors are, characteristically speaking, slow growing and include low-grade micropapillary serous carcinoma, mucinous, and endometrioid carcinomas. Type I tumors are also genetically stable and are characterized by mutations in a number of different genes including KRAS, BRAF, pTEN, and beta-catenin. Type II tumors, however, are rapidly growing and highly aggressive neoplasms for which well-defined precursor lesions have not been described. Type II tumors include high-grade serous carcinoma, malignant mixed mesodermal tumors (carcinosarcomas), and undifferentiated carcinomas and have a high level of genetic instability and are characterized by a mutation of TP53. This method and assay disclosed herein, helps to explain why current screening techniques, aimed at detecting stage I disease, have not been effective given that Type II tumors are rarely confined to the ovary and are not derived from Type I EOC. Current screening approaches for early EOC detection have utilized stage I cancers as model in early markers development. However, specifically in regard to Type II EOC, such screening approaches and models are flawed for the reasons discussed above. The diagnostic methods disclosed herein, however, provide an improved, novel, and unique method allowing for earlier diagnosis of Type II EOC.

Ascitic fluids are representative of an EOC tumor microenvironment and/or peritoneal washings (for those patients who do not develop ascites or are still at a relatively early stage of disease development) which can be obtained through minimally invasive procedures including ultrasound guided procedures. Accordingly, the PLA₂ activity based methods provided herein provide a ready and less invasive means for diagnosing and monitoring the progress of these diseases than those in current use.

Additionally, the single blood marker disclosed herein may be used as part of an array of diagnostic tools and has important value to existing EOC markers such as the markers marketed by Quest Diagnostics (OVAL Ca¹²⁵ and HE4).

The study reported herein investigated EOC ascitic and tissue cPLA₂- and iPLA₂-like enzymatic activities. This study included validating the quantitative nature of the DBPC-based PLA₂ assays via extensive trials. These studies show the presence of microvesicle-free cPLA₂ and iPLA₂ in human EOC ascites as evidenced by PLA₂ enzymatic activity assays. This unexpected finding was made by carefully measuring the sensitivities of ascitic tumor-promoting activities to PLA₂ selective inhibitors, and by measuring lipid products produced via the activities of these enzymes.

It is understood that all pharmacological inhibitors have potential off-target effects. For example, BEL has been shown to have non-iPLA₂ targets. Genetic tools such as RNAi techniques in cell-free fluids are not available to confirm those results, however, several lines of evidence support the conclusions made herein, these results are as follows: 1) the DBPC-based PLA₂ assays eliminate most off-target effects, since other known BEL targets do not use DBPC as a substrate; 2) the lack of divalent cations in the presence of EDTA further specifies iPLA₂ activity; and 3) the cell-based assays and lipid production assays used herein consistently support the cPLA₂ and iPLA₂ activities. When applicable, genetic methods, such as siRNAs against LPA receptors, were used to confirm the results.

It was found that the ascites exosome fraction (P3) had low or no tumor-promoting activity in vitro. In contrast, it was found that the ultracentrifuged, exosome-free S4 fraction (similar to the cell-free S1 fraction) had strong tumor promoting activity both in vitro and in vivo, these measurements suggest that the majority of the tumor-promoting activities present in ascites are in an exosome-free form. The ascitic tumor promoting activities were correlated with and functionally related to PLA₂ activity. Inhibitors against ABCC1 and ABCB1 (Pgp) reduced secreted MAFP-sensitive, EDTA-partially insensitive PLA₂ activity in CM from RAW 264.7 mouse macrophages, suggesting that these transporters are involved in the secretion pathway of PLA₂ enzymes from these cells. Interestingly, the drug resistant SKOV3-VLB cells (overexpressing ABCB1) responded similarly to the parental cells to the migration/invasion-promoting activity of ascites fractions, suggesting that the same targets/signaling pathways may also be effective in drug-resistant EOC cells. These novel findings warrant further study to characterize the cellular source of the cPLA₂- and iPLA₂-like activity in EOC ascites and the secretion mechanisms involved. Still another unexpected finding is that MAFP inhibition could effectively block the decrease in LPC and the increase in LPA, but not the increase in arachidonic acid. cPLA₂s and iPLA₂s also use substrates (mainly PCs) containing other fatty acids. It is possible that the alterations of one and more other fatty acids are more correlated with LPC changes. In addition, the levels of arachidoinic acid can be regulated by many other enzymes; these results suggest that its levels in ascites are not affected by MAFP.

As demonstrated by the inhibitor and siRNA work presented here, LPA present and produced by ATX and PLA₂ in 51 and S4 ascites fractions mediates a significant part of the tumor-promoting effects of ascites as evidenced by ex vivo LPA production in 51 and S4 ascites fractions. While 40-50% of the effect of ascites fractions on EOC cell migration is heat stable, an LPA antagonist inhibits more than 70% of the effect. The heat-stable effect was likely due to the lipid signaling molecules (LPA in particular) that are already present in the ascites. These results imply that lipids that are continuously synthesized in ascites via PLA₂s and ATX are functionally involved in the biological effects and the enzymes are heat-sensitive. In addition, protein factors (likely to be heat-sensitive) can modulate LPA signaling. For examples, LPA in biological fluids binds to serum albumin and other proteins that enhance its effects on cellular functions. EGF and angiotensin II modulate LPA₁ receptor function and phosphorylation state. EGF increases LPA production in human EOC cells. Therefore, LPA receptor blocking resulted in a higher percentage of inhibition. Published data on catabolic loss of LPA via lysolipid phosphatases and other enzymes, as well as local and temporal supplies of signaling LPA generated by ATX, strongly support the notion that dynamic processes are involved in both production and degradation of bioactive lipids in the tumor microenvironment. Interestingly, while is has been shown that each of LPA₁₋₃ play important roles, LPA₄ is not likely to be involved in S1- or S4-induced cell migration, consistent with the reported inhibitory effect of LPA₄ on migration of mouse embryonic fibroblasts, colon cancer cells, and neuroblastoma cells.

Unexpectedly, PLA₂ activities are elevated in EOC tumor tissues. Indeed preliminary data shows that PLA₂ activity is also elevated in the blood of EOC patients (data not shown). These data imply that PLA₂ activity may represent a useful marker for diagnosis, prognosis and/or disease monitoring of EOC. Regardless of whether PLA₂ enzymes contribute indirectly (by providing substrate for ATX) or directly (by using PA as their substrate), these data show that PLA₂ enzymes play an important role in producing oncogenic LPA and hence are an important target in EOC. Taken together, these results have provided provocative and heretofore unrecognized directions for developing new therapeutic modalities for EOC.

Further, tissue based PLA₂ activity assays, as disclosed herein, offer information related to tumor and host cell histology, and potentially better separation for subjects in different groups. DBPC is used in assays, disclosed and described herein, for in vitro diagnostic applications. Additionally, development of infrared fluorescence (NIRF) PLA₂ substrates suitable for in vivo tumor imaging provide for in vivo EOC imaging. Utilization of the PLA₂ activity assays, disclosed herein, for monitoring PLA₂ activity provides not only available diagnostic tool, but also an efficient means of monitoring the efficacy of various treatments including drug treatments.

The data presented herein provides and supports a method of using PLA₂ activity as a new marker for studying a disease, such as EOC, including methods of diagnosing the diseases, predicting the course of the diseases, evaluating the effectiveness of treatments for the diseases and formulating personalized treatment regimes for this disease. As discussed herein, PLA₂ activity provides a useful marker for diagnosis, prognosis, monitoring, and/or predicting EOC and/or other GYN cancers. These methods are especially useful when PLA₂ activity is normalized to an inherent standard such as the number of cells in a sample of tissue. Still other means of normalizing PLA₂ activity include normalizing data based on the protein content of a given sample and the like. The data indicated herein further provides that PLA₂, including iPLA₂ alone or in combination with other PLA₂s provides a useful target for the treatment of EOC. Further, PLA₂-related activities, disclosed herein, are highly significant in EOC management and treatment decision making, as well as in designing and testing new therapeutics. Further, it should be understood that although genetic, epigenetic, and proteomic studies regarding EOC have been conducted, those studies have focused on expression at either the RNA or protein levels, not a direct focus on PLA₂ activity. Thus, a focus directly on PLA₂ as a marker for ovarian and other gynecological forms of cancer, as disclosed herein, represents a new and highly relevant and useful method for the diagnosis, monitoring, and targeted treatment of gynecological cancers such as EOC.

### EXPERIMENTAL

### Example 1

### Detection Assays for PLA₂ Activity

Classical methods of assaying PLA₂ activity, in general, involve the use of a radioactive-labeled phosphatidyalcholine (PC) substrate. Cell or tissue lysates are incubated in a buffer with or without calcium (assays specific for a form of iPLA₂ are performed without calcium and include 1 mM EDTA) and the substrates [dipalmitoyl phosphatidylcholine (DPPC), and 1-palmitoyl-2-[1-¹⁴C] palmitoyl-sn-glycero-3-phopshocholine (300,000 cpm/assay)]. Post incubation, the cleaved and ¹⁴C-labeled fatty acid (FA) is resolved using thin-layer-chromatography (TLC). The radioactivity associated with the cleaved FA is counted and calculated. However, this standard assay is not convenient or practical for high throughput PLA₂ activity assays.

According to the instant disclosure, a PLA₂ activity assay practical for high throughput utilizing DBPC (available from Echelon Biosciences Incorporated, Salt Lake City, Utah 84108 Echelon Biosciences Incorporated, Salt Lake City, Utah 84108) is disclosed. DBPC is a fluorogenic Dabcyl- and BODIPY-containing PC which functions as a PLA₂ substrate producing fluorescence. Usages of DBPC has the advantage of not involving radioactivity or a time-consuming and labor intensive TLC separation step. Further, the assay disclosed herein has been adapted for use in frozen tissue sections with cell nuclei co-stained to examine the types of cells, having significant PLA₂ activity, and tissue histology, simultaneously.

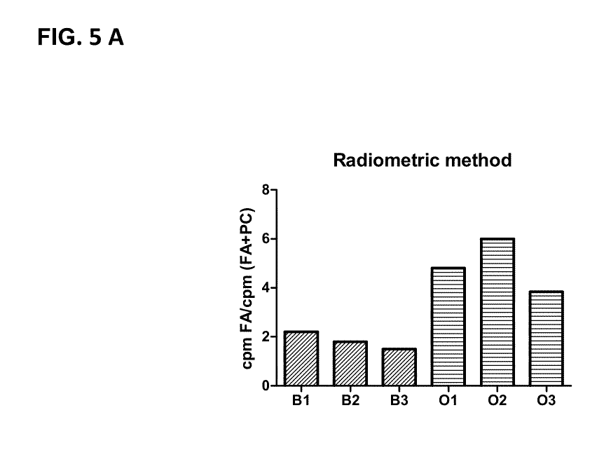

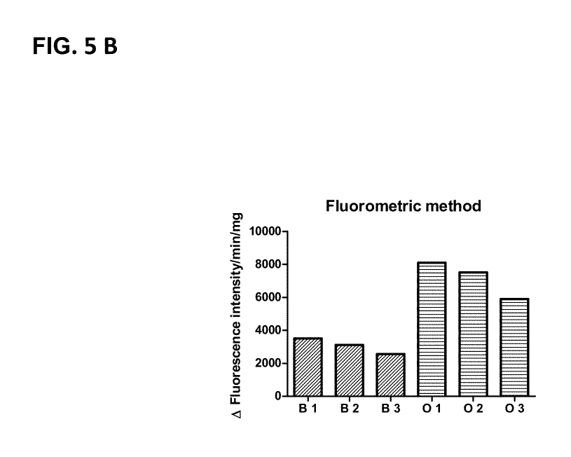

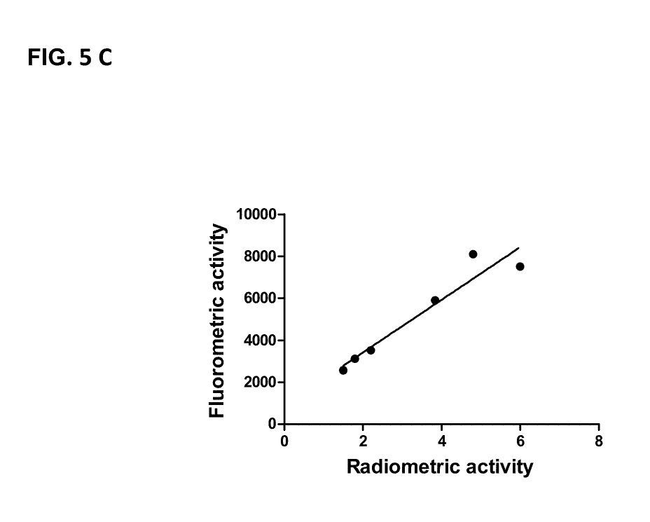

DBPC has been used in cell based PLA₂ assays. However whether it could be used for quantitative assays in tissues or biological fluids had not been tested. As reported herein, an assay validated DBPC-based quantitative PLA₂ assays in human samples (tumor tissues and ascites). Referring now to FIG. 5, first PLA₂ activities in tissue homogenates (ovarian tumors vs. benign gynecologic tumors, n=3 each) were compared using both the DBPC and the classical radiometric method (6, 16, 23). The results were highly correlative despite the different arbitrary units used for activity, supporting the quantitative nature of the DBPC method.

Next, the conditions to be used for DBPC assays were optimized. It was found that the increase in fluorescence was linear for at least 4 hr incubation at 37° C. A 2 hr end-point was chosen for most assays reported here. It was also found that DBPC was sensitive to freeze-thaw cycles with decreased fluorescence. However, after 3 freeze-thaw cycles, it became stable when stored at −80° C. Hence, aliquoted and freeze-thaw treated DBPC substrate were used for the assays. The precision and reproducibility of the assays was examined using three human EOC ascites samples. The intra-day coefficient of variation (CV) was between 3.7 to 5%. Similarly, the inter-day CVs were less than 3.2% for all samples tested. Using two different ascites samples, it was found that 1-3 freeze-thaw cycles did not significantly change the PLA₂ activity. In addition, the PLA₂ activity was not changed in samples stored at −80° C. for at least 4 months, suggesting that the activity was relatively stable. The amount of substrate to be used was also optimized. Using a fixed amount of ascites (10 μL), it was determined that the increase in fluorescence was linear at least in the range of 0.1 to 0.6 μg DBPC. A 0.2 μg DBPC substrate was used in those assays. Those assays identified the linear range for each type of biological sample to insure that the substrate would not be rate-limiting. The linear ranges were 0.2-1 μL and 0.002-0.02 mg protein for ascites and tissues homogenates, respectively. Thus, the optimized conditions for the work presented here were: 0.2 μg of DBPC and 1 μL of ascites or 0.02 mg protein of tissue homogenate in 200 μL PBS for 2 hrs incubation at 37° C. The PLA₂ activities were expressed as change in fluorescence intensity/min/4 or as change in fluorescence intensity/min/mg of protein. Although the protein concentrations of 10 human EOC ascites specimens that were used in this work ranged from 39-64 mg/mL, there was no correlation between PLA₂ activity and the total protein concentration in ascites (R²<0.01, P>0.5), suggesting that PLA₂ activity is an independent parameter of ascites (Table 3).

TABLE 3
Protein concentration, PLA₂ and ATX activities of ascites samples.

| | protein | natural | | | | | |
|---|---|---|---|---|---|---|---|
| ID | (mg/mL) | PLA2 ^{#} | iPLA2 ^{#} | sPLA2 ^{#} | cPLA2 ^{#} | ATX ^{#} | ATX/BrP-LPA ^{#} |
| A1 | 61 | 378 | 434 | 0.9 | 269 | 27.0 | 7.9 |
| A2 | 45 | 466 | 558 | 5.1 | 407 | 31.4 | 6.8 |
| A3 | 42 | 213 | 294 | 2.0 | 188 | 31.4 | 8.0 |
| A4 | 39 | 319 | 452 | 5.4 | 184 | 28.6 | 5.2 |
| A5 | 48 | 213 | 331 | 0 | 132 | 29.4 | 5.2 |
| A6 | 55 | 247 | 382 | 7.4 | 181 | 35.7 | 6.6 |
| A7 | 54 | 384 | 595 | 3.7 | 300 | 39.2 | 7.6 |
| A8 | 47 | 381 | 495 | 5.2 | 273 | 30.0 | 9.3 |
| A9 | 48 | 225 | 369 | 2.2 | 166 | 43.0 | 9.8 |
| A10 | 64 | 302 | 463 | 6.3 | 207 | 33.5 | 5.6 |

### Example 2

### PLA₂ Activity Assay Using Tissue

PLA₂ activity from human gynecologic (GYN) cancers (including both EOC and endometrial), benign GYN disease, and normal tissue samples were tested. Six samples from each of these groups were assayed in accordance with the instant disclosure.

Tissue samples were obtained and frozen in order to preserve enzymatic activity. The tissue samples (of average size 6-10 μm) were fixed using acetone and methanol at −20° C. according to typical fixation methods known within the art. As explained above, the substrate DBPC is a fluorogenic Dabcyl- and BODIPY-containing PC which represents PLA₂ activity with fluorescence.

The PLA₂ activity in the frozen tissue sections was quantified. Quantification of the PLA₂ signals in frozen tissue sections used the software (MetaMorph). The total integrated signaling intensity, with the background subtracted (tissues processed the same way in the absence of the PLA₂ substrate), were captured and measured, and then normalized based on cell number or per cell ratio (the cell numbers were counted by DAPI-stained nuclei).

Referring now to FIG. 1, the activity of PLA₂ is shown illustrating PLA₂ activity being elevated in cancer (FIG. 1A) vs. benign (FIG. 1B) and normal (FIG. 1C) GYN tissue samples. Representative tissue PLA₂ activities are shown. The first two rows in FIGS. 1A to C, (each comprising four panels labeled as lower case letters a to d) present: a.) no DBPC substrate; b.) addition of DBPC; c.) DAPI staining of cell nuclei; and d.) an overlay the DAPI and PLA₂ activity staining Referring now to FIG. 1D, para-nuclear PLA₂ activities are demonstrated as being sensitive to inhibitors of iPLA₂ and cPLA₂ (e.g., AACOCF3 and BEL) but not to a inhibitor of sPLA₂ (e.g., TA-PC) (the big bright spots were present in the absence of DBPC and thus were non-specific and were not a PLA₂ activity). Remaining with FIG. 1D, the numbers in each panel (a to d) are the activities expressed as fluorescent intensity/cell under each condition (as described herein).

Referring now to FIG. 1E. FIG. 1E presents PLA₂ activities measured in six samples from each of cancer (EOC and endometrial), benign GYN, and normal groups. Panel a of FIG. 1E presents individual values; whereas panel b of FIG. 1E presents mean and SD values (**, P<0.01; ***P<0.001).

As shown herein, PLA₂ activities in EOC samples were significantly increased compared to those in either benign or normal tissues. The PLA₂'s activities/cell in the three groups of samples were 881+/−395; 276+/−58; and 55+/−16 for human cancer, benign, and normal tissues, respectively (FIG. 1). The results presented herein illustrate that high levels of para-nuclear PLA₂ activities in the cancer tissues were only observed in the ovarian and endometrial cancer tissues (FIG. 1A). For benign tumors, these activities were limited to the superficial cell layers of the benign tumors (FIG. 1B). For normal tissues, very little or no para-nuclear PLA₂ activity was observed (FIG. 1C). The long-thin bright lines detected in some normal tissues (FIG. 1C) are unlikely to be cellular PLA₂ activities, since they were insensitive to any of the PLA₂ inhibitors tested (data not shown). In contrast, cancer PLA₂ activities were sensitive to inhibitors for both cPLA₂ and iPLA₂ (e.g., AACOCF3 and BEL) but not to TA-PC (an inhibitor of sPLA₂) (FIG. 1D). Thus the data presented herein illustrates that a majority (if not the total PLA₂ activity tested in tumor tissues) is derived from cPLA₂ and/or iPLA₂. It is worth noting that the individual values of PLA₂ activity from different groups were not overlapping, indicating this assays excellent separation and high specificity (FIG. 1E, panel a). As disclosed and described herein, a cut-off or determination value for identifying tumor tissues from both benign and normal tissues is approximately 400 (normalized fluorescent intensity values/cell). A determination or cut-off value of 400 (normalized fluorescent intensity values/cell), as disclosed herein, provides a 100% specificity in separating the various tissue states tested and described above.

### Example 3

### PLA₂ Activity Assay Using Tissue Homogenates

Further, a tissue homogenization method, in which tissue lysate is utilized for measuring PLA₂ activity, is disclosed herein. The homogenization method described herein was compared against the current standard quantitative methods of tissue using a radioactive assay (as discussed above), for assessing quantification using tissue homogenates for measuring PLA₂ activity. As described herein, a tissue homogenization method suitable for PLA₂ activity analysis was developed.

According to the instant disclosure, frozen samples where pulverized and transferred to a microcentrifuge tube and mixed with 500 μL lysis buffer. It is within the instant disclosure that samples may be pulverized in any known method (including using pulverizing instrumentation, bead ablation, and manual grinding). The lysis buffer comprised 10 mM hepes, pH 7.5, and 0.34 M sucrose with an additional 54 of mammalian protease inhibitor cocktail obtained from Sigma-Aldrich, Inc. The tissue samples were next homogenized using tissue homogenization instrumentation (such as the Omni Tissue Homogenizer, Model No.: LR60902). Following lysis and homogenization, the samples were centrifuged at 16,000×g, at a temperature of 4° C., for about 40 minutes. Following centrifugation the supernatants were transferred to a new eppendorf tube for assaying the PLA₂ activity.

PLA₂ activities measured using the standard radioactive methods (discussed above) were 1.76, 1.30, 0.52, and 0.57 [Fatty acid/(Fatty acid+PC)]%] (normalized to the same amount of protein (1 mg) in each sample) for the two cancer samples and two normal samples, respectively. Taken together, these data show that EOC and possibly other forms of GYN cancers have elevated PLA₂ activities when compared to those of benign GYN and normal tissues.

### Example 4

### PLA₂ Activity in Ascites and LPA Levels in Plasma

The method described herein, is also adaptable for use in identifying PLA₂ activity as a marker in body fluid. For reasons discussed in detail above, a body fluid PLA₂ activity marker provides a more convenient and clinically useful marker, than a genetic number marker or a marker based on RNA.

The PLA₂ activity detection method employed herein is simple and can be easily conducted in almost any clinical laboratory. In contrast, although LPA has been shown to be a potential EOC marker, because of obstacles such as low concentration in the blood, variable samples processing procedures, as well as the rather sophisticated (less clinic-friendly) and different electrospray ionization tandem mass spectrometry (ESI-MS/MS) methods used in it, has proven slow in moving to wide spread and accepted clinical use.

Those results show that EOC ascites contained high levels oncogenic lipid growth factors, such as LPA, that stimulate EOC cell proliferation, adhesion, migration, and invasion. Compared to ascites from patients with benign liver cirrhosis, EOC ascites also contained higher levels of lysophosphatidylcholine (LPC), the lipid product of PLA₂. Arachidonic acid (AA, another product of PLA₂ activity) was analyzed and it was found that it was also high in EOC ascites (3.2±1.4 μM and 0.90±0.43 μM for EOC and liver cirrhosis ascites samples, respectively, n=10 in each category, P<0.001). The combined elevation of LPC/LPA and AA support high PLA₂ activities in EOC ascites.

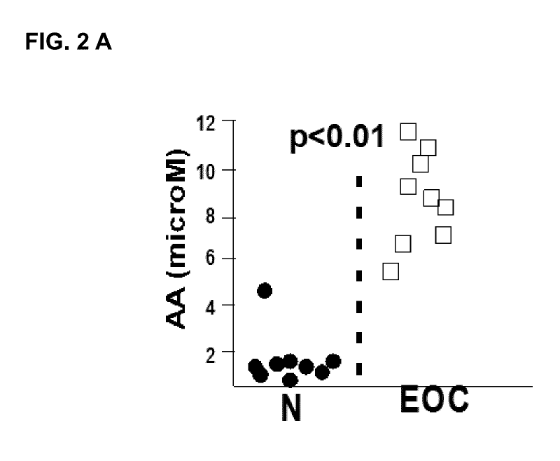

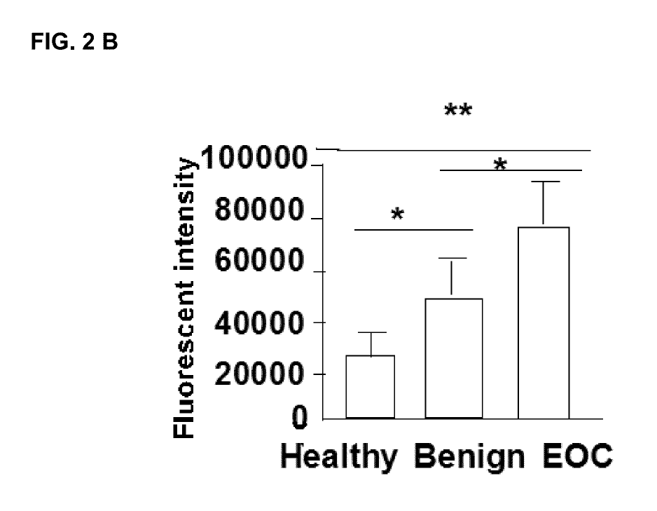

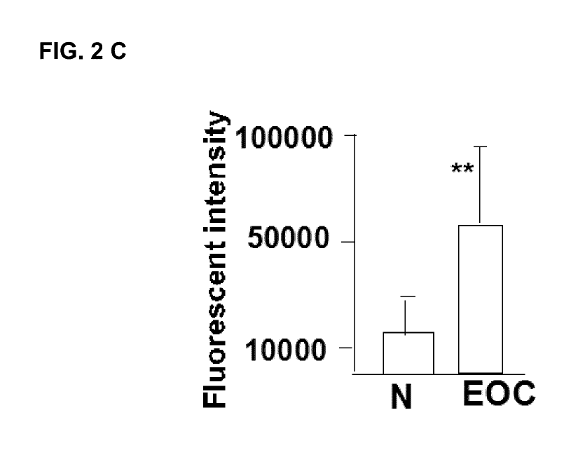

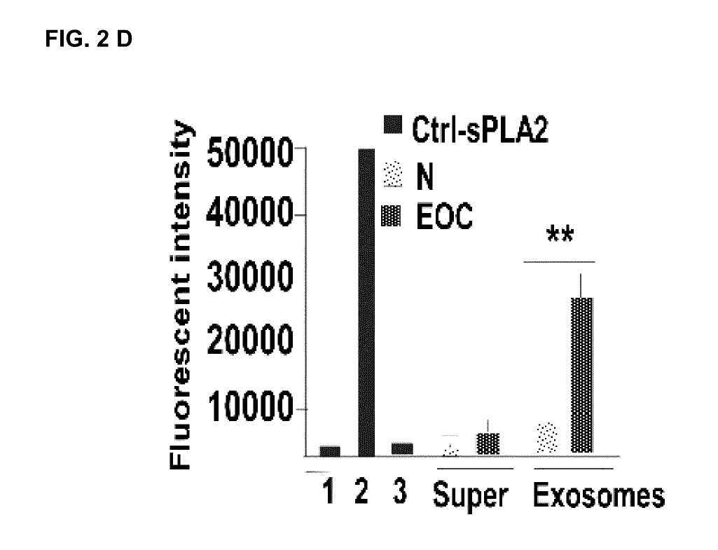

Referring now to FIG. 2B, the dark bars: 1. background fluorescence; 2. sPLA₂ positive control; 3. TA-PC treated sPLA₂. **: p<0.01. *p<0.05. The total PLA₂ activities in blood samples from healthy (n=7) subjects, benign (n=8) subjects, and subjects diagnosed with EOC (n=5). as reported herein, PLA₂ activity is increased, with a concurrent increase in PLA₂ direct and/or indirect products (such as LPC, LPA, and arachidonic acid (AA)) in EOC ascites and blood samples. Data measured with LPC and LPA have been published, and increased LPA in EOC blood samples has been confirmed in repeated and independent studies.

According to the present disclosure, PLA₂ activity in human ascites samples were collected and assayed for detection of DBPC derived fluorescence. According to the disclosed method, ascites samples (25 μL per sample) were used in assessing PLA₂ activities. The fluorescence detected (shown as total intensity in the accompanying figures) were approximately 2400 and 4,000 (wherein the numbers in the figures should be divided by 25, respectively, based on sample volume). The background fluorescence (intensities/4) in the human samples assayed, according to the method disclosed herein was observed to be relatively low (˜40) and did not change in a considerable amount during a 24 hour incubation time.

According to the methods disclosed and described herein, human ascite samples were centrifuged at 3,000 g for 20 minutes at 4° C. The samples were then aliquoted and stored at −80° C. Ascite samples (25 μL of each) were suspended in 50 μL buffer [comprising 80 mM hepes (pH 7.4), 150 mM NaCl, 10 mM CaCl₂, 4 mM Triton X-100, 60% glycerol, and 1 mg/mL BSA], respectively, and then mixed with 0.20 μg DBPC [dissolved in 50 μL DMSO/assay buffer (1:100)]. The total volume of each assay was approximately 200 μL. After incubation for 4 hrs, fluorescent intensities were measured at 485 nm/535 nm in a plate reader (Perkin Elmer Victor³V 1420 Multilabel Counter).

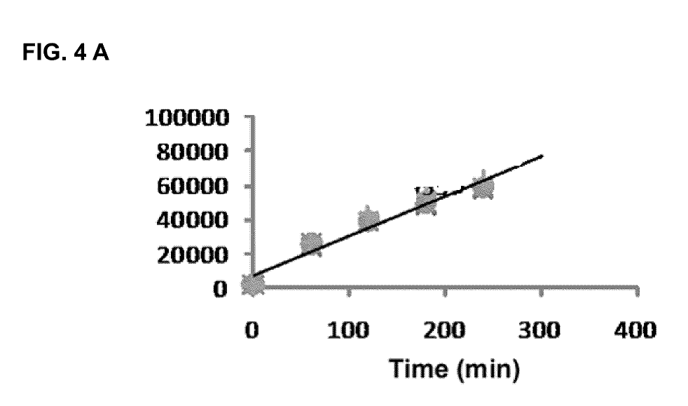

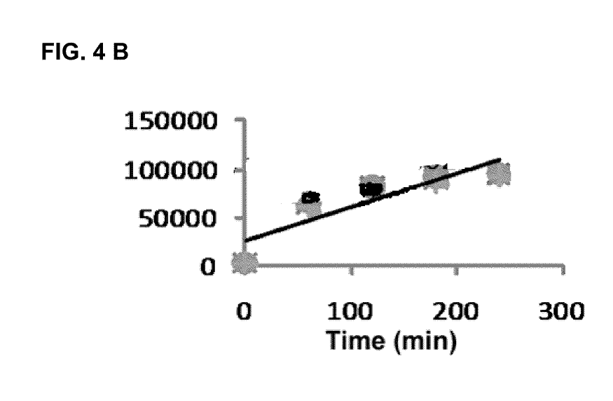

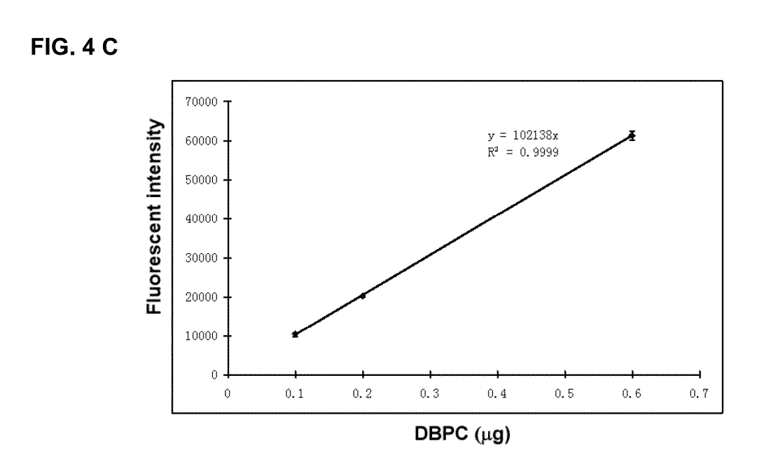

Referring now to FIGS. 4A and B, Two time-dependent curves are presented demonstrating fluorescence observed having a linear relationship with time over a 4 hour assay time period for different samples. The data in FIG. 4 A fit to a line with the following values: Y=230, intercept of 7468 and a R squared value of 0.9559. The data in FIG. 4 B fit to a line with the following values: Y=352, intercept of 25,328 and a R squared value of 0.7818.

With reference now to FIG. 4C, the method and assay disclosed herein is demonstrated as comprising good inter-day and intra-day precision with all assays performed. FIG. 4C, presents a standard curve which correlates intensity value of assayed EOC ascite samples with DBPC concentrations used.

As shown by the methods described and disclosed herein, PLA₂ is associated with human EOC ascites and exosomes (from ascites and blood). Both cPLA₂s and iPLA₂s are cytosolic enzymes, only sPLA₂s are secreted, data presented herein shows that elevated PLA₂ activity (which may be due to the activity of any of these three classes of PLA₂s) can be detected in the cell-free blood and/or ascites samples from patients with EOC and/or other GYN cancers.

Exosomes are 40-100-nm diameter membrane vesicles released from multivesicular bodies (MVB) by intact cells and are thought to participate in intercellular signaling. Further, cell membrane lipids, including PC (a PLA₂ substrate) and sphingolipids (in particular ceramides) are prominent lipids within exosomes.

According to methods of the instant disclosure, the ExoQuick kit from SBI System Biosciences (which has been validated to be more effective in exosome isolation than the more traditional ultra-centrifugation method, available at http://www.systembio.com/exoquick-exosomes/) was utilized in isolation of exosomes from human ascite samples. As demonstrated by the instant disclosure, significant increases in PLA₂ activities were detected in EOC blood, cell-free ascites, and exosomes vs. similar samples collected from non-malignant liver diseases or healthy controls. With reference to FIG. 2, approximately 80% of cell-free ascitic PLA₂ activity is shown as associated with the exosome portion in ascites. In addition, the observed exosome-associated PLA₂ activity proved sensitive to methyl arachidonyl fluorophosphonate (MAFP) (an irreversible dual cPLA₂ and iPLA₂ activity inhibitor), BEL (an irreversible and selective iPLA₂ activity inhibitor), and/or thioether amide-PC (TAPC) (a selective sPLA₂ activity inhibitor). Referring now to FIG. 2B, the inhibitor's effectiveness is confirmed by its ability in blocking sPLA₂ activity when a positive control (from bee venom; Cayman) was used (FIG. 2D). As such, the method and disclosure herein, provides analysis of PLA₂ activity, which is demonstrated herein to be associated with human EOC ascites and exosome (from ascites and blood), presents a novel and highly useful method for diagnostic and personalized treatment purposes in GYN cancers.

### Example 5

### cPLA₂- and iPLA₂-Like Activities Account for the Majority of the PLA₂ Activities Detected in Cell- and Vesicle-Free Human EOC Ascite

Lucas and Dennis have summarized the inhibitors and assay conditions for distinguishing the different PLA₂ subtypes. Similarly, a set of conditions the assay conducted in this study were selected to distinguish PLA₂ activity derived from different subtypes: a) the “natural PLA₂ activity” was detected in the samples without any exogenous additives, b) the iPLA₂ activity was detected in the presence of 5 mM EDTA (a divalent cation chelator to block all PLA₂s requiring calcium, including sPLA₂ and cPLA₂), c) the sPLA₂ activity was detected in the presence of 1.2 mM calcium chloride (the natural ionized calcium concentration in blood (25)) and MAFP (10 μM, a dual inhibitor of cPLA₂ and iPLA₂), and d) the cPLA₂ activity was detected in the presence of 100 μM calcium chloride and bromoenol lactone (BEL, 10 μM, a selective inhibitor for iPLA₂).

Strong PLA₂ activity was detected in all 10 human EOC cell-free ascites specimens tested. While sPLA₂ has been described in cell-free biological fluids, surprisingly it was found that cPLA₂- and iPLA₂-like activities (MAFP-sensitive) accounted for >98% of the “natural PLA₂ activity” detected in cell-free ascites (Table 3). The sum of defined cPLA₂ and iPLA₂ activities were higher than the “natural PLA₂ activity”. This is likely due to enhanced iPLA₂ activity beyond its “natural activity” in biological samples under the conditions used. In addition, cautions were taken in interpretation of the results with inhibitors, since their efficacies and specificities may not be 100% and they may have off-target effects. Additional reagents, such as thioether amide-PC (TAPC), an sPLA₂ selective inhibitor, DTT (reduces disulfide bonds to denature protein structures of sPLA₂s), or LY311727 (a selective inhibitor for sPLA₂ II) did not inhibit the ascitic PLA₂ activity, confirming the low sPLA₂ activity in ascites.

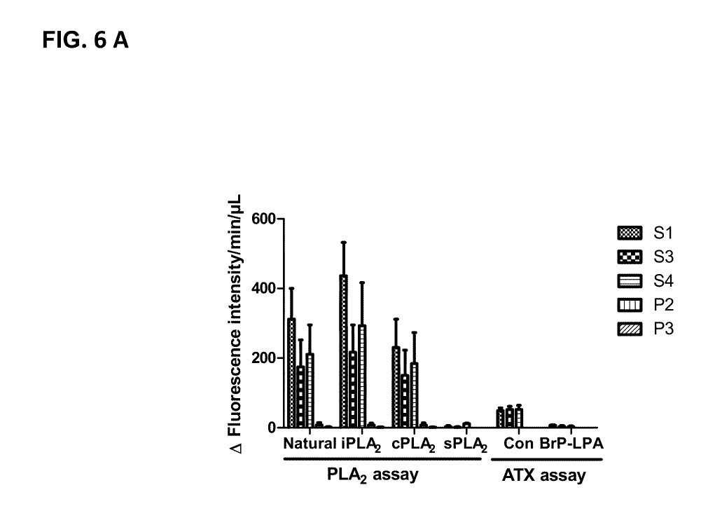

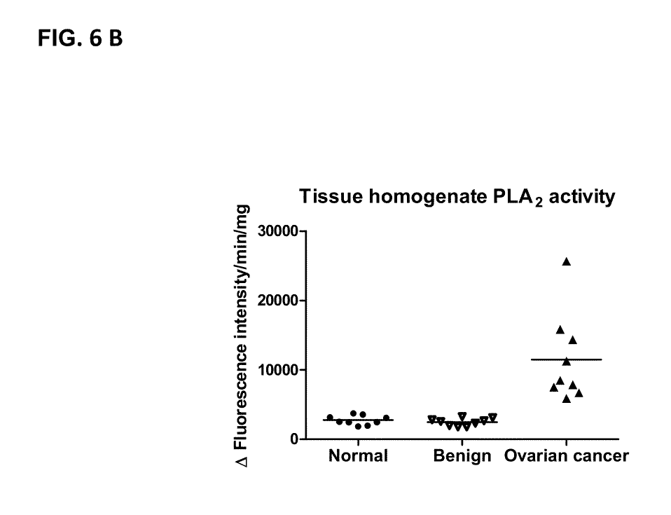

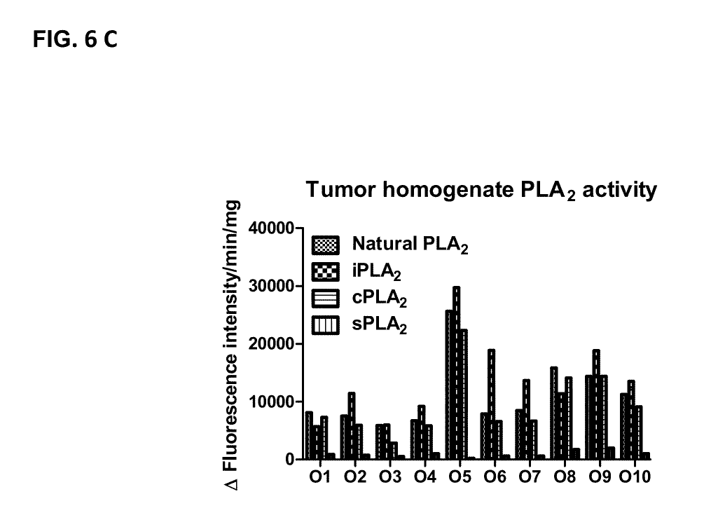

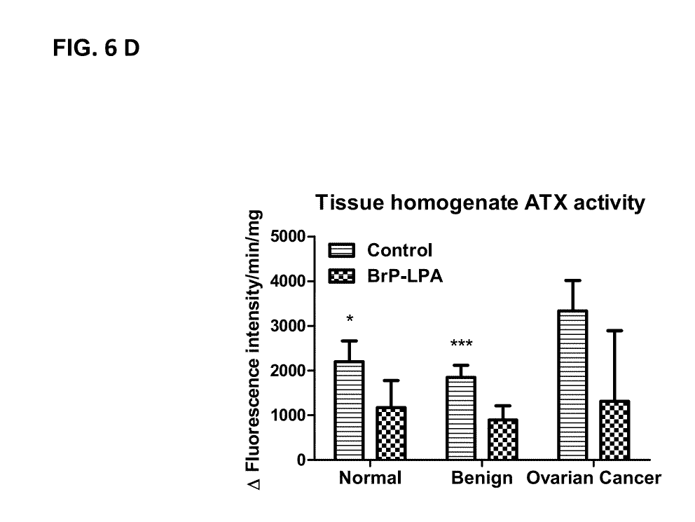

cPLA₂ and iPLA₂ are cytosolic enzymes and not known to be secreted by classical secretion pathways. It has been reported that PLA₂ activity is associated with reticulocyte and mast cell exosomes. To test whether EOC ascitic PLA₂ activities were associated with exosomes, step-wise centrifugation was used to obtain exosomes. As shown in FIG. 6A, it was found that >50% of the natural PLA₂, iPLA₂, and cPLA₂ activities remained in the S3 and S4 fractions of the ascites, and <3% of these activities was associated with the P3 (exosome) fraction when the activities were adjusted to the original volumes. The P2 fraction (cell fragments and some larger vesicles) contained <3% of PLA₂ activities as well (FIG. 6A). Highly purified exosomes can be obtained from P3 using a sucrose gradient. However, since minimal PLA₂ activities were associated with P3, additional work with P3 and more purified exosomes were not the major focus of the current work.

It has been demonstrated that LPA is a major lipid growth factor for EOC and ATX has been considered the main or even the sole enzyme producing LPA in biological samples. Strong ATX activity was detected in all 10 EOC ascites samples tested (Table 3). Minimal or no ATX activity was associated with P2 or P3 fractions. All ATX activity detected in the 51 fraction was maintained in the S3 and S4 fractions and >85% was inhibited by the ATX selective inhibitor BrP-LPA (100 μM) (FIG. 6A).

### Example 6

### PLA₂ Activity in Mouse Tissue

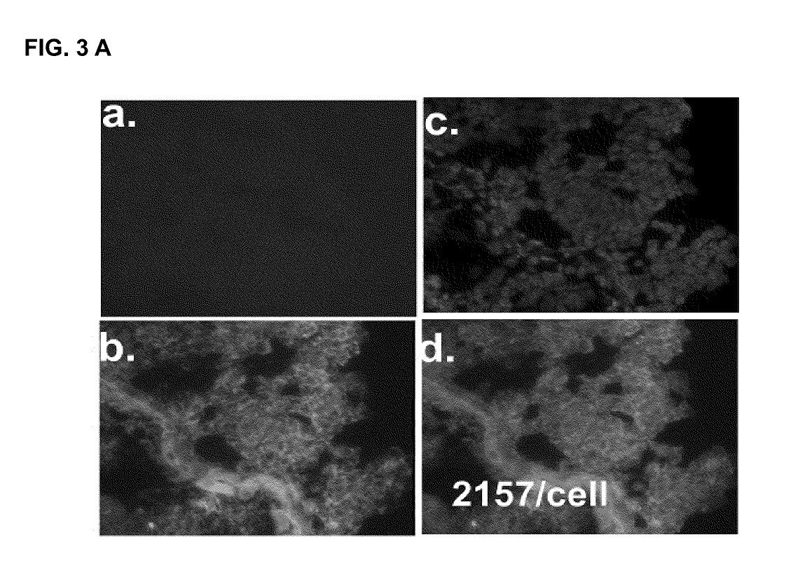

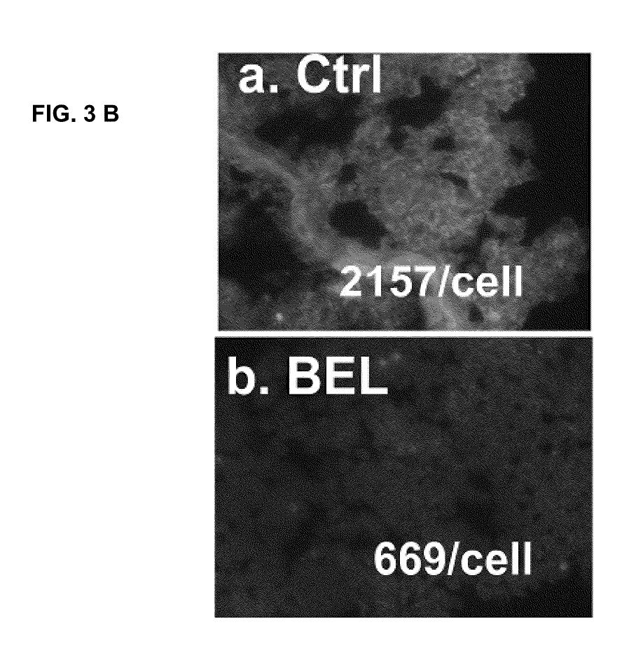

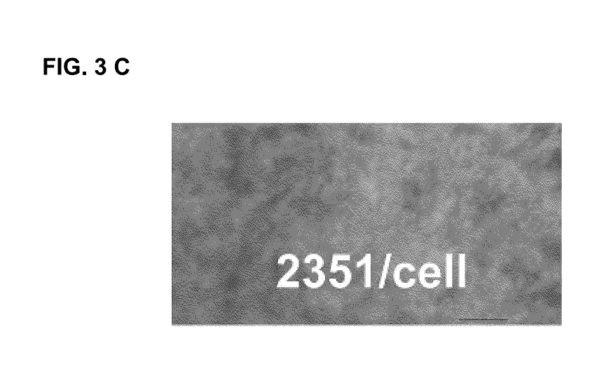

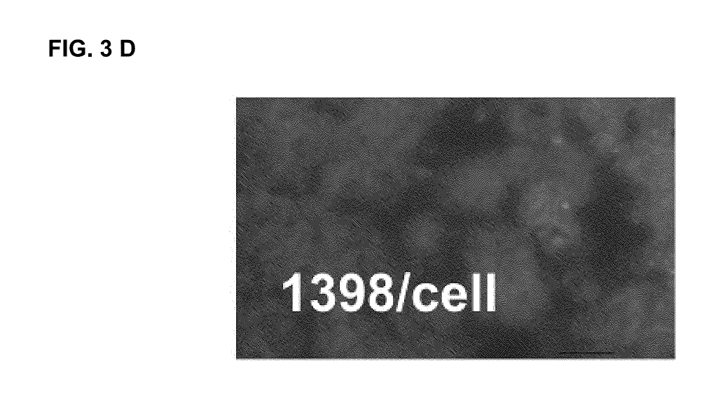

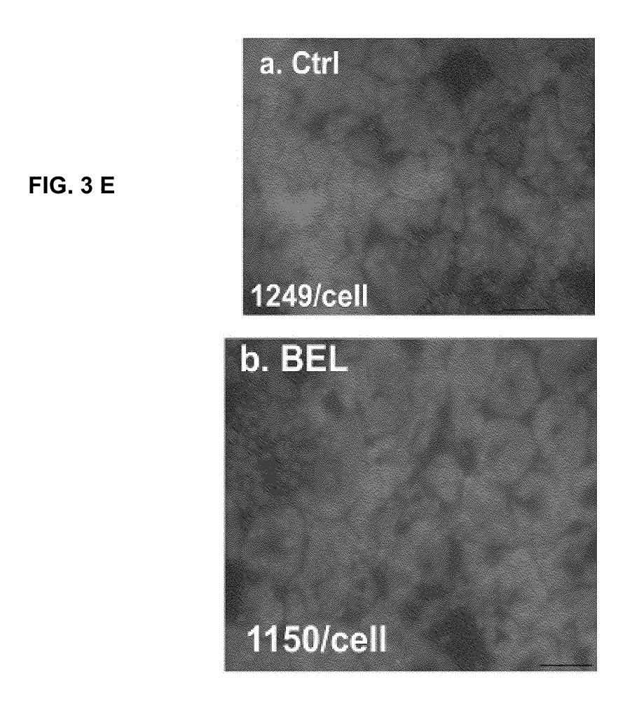

Referring now to FIGS. 3A, C and D, PLA₂ activities measured from several mouse models for GYN diseases are presented. These assays demonstrate high PLA₂ activities in tumors from human A2780 and SKOV3 xenografts, as well as from tumors derived from mouse EOC ID-8 cells. Further, and as shown herein, PLA₂ activity is greatly reduced in tumors from bromoenol lactone (BEL)-treated mice compared to those from controls. Further, as shown herein, BEL targets PLA₂ enzymes (see, FIG. 3B, panels a-b) and reduces tumorigenesis, correlating to a reduction in PLA₂ activity as reducing tumorigensis.

Referring now to FIG. 3E, PLA₂ activity was measured in several other mouse tissues, including the kidney, lung, and liver. Tumors did not grow in or on these organs, and tumor cell injections did not significantly change the PLA₂ activities in these organs (data not shown). Further, it was determined that at least some (and possibly most) of the activities detected in these tissues appeared to be sPLA₂ activity of normal tissues (based on the activity's extracellular localization and insensitivity to BEL and AACACF3). In all experiments, controls were run which did not include added substrate. Only the merged pictures and only kidney results among different tissues are shown in FIGS. 3B to 3E (some data not shown). These data demonstrate the use of PLA₂ activity for following progression of these diseases and/or to monitor the effective of various treatments for the disease.

### Example 7

### cPLA₂ and iPLA₂ as Well as ATX Activities were Elevated in EOC Tissues

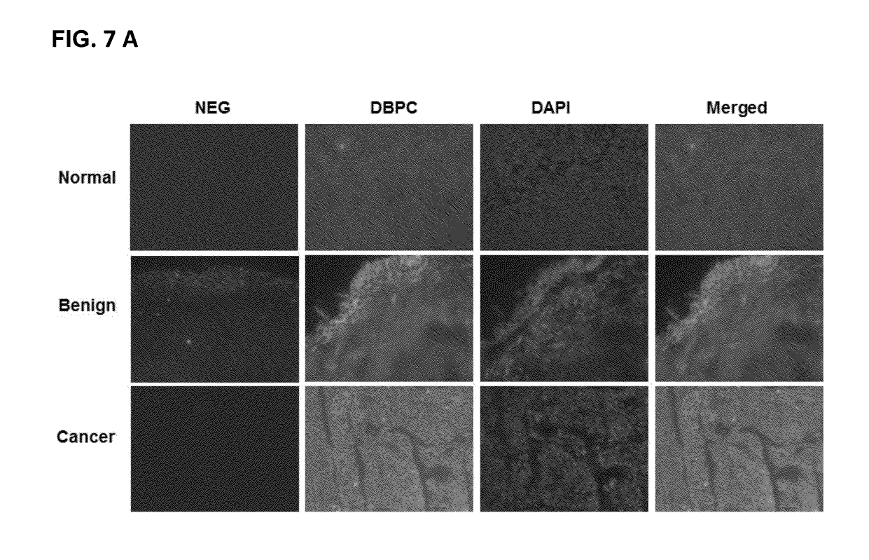

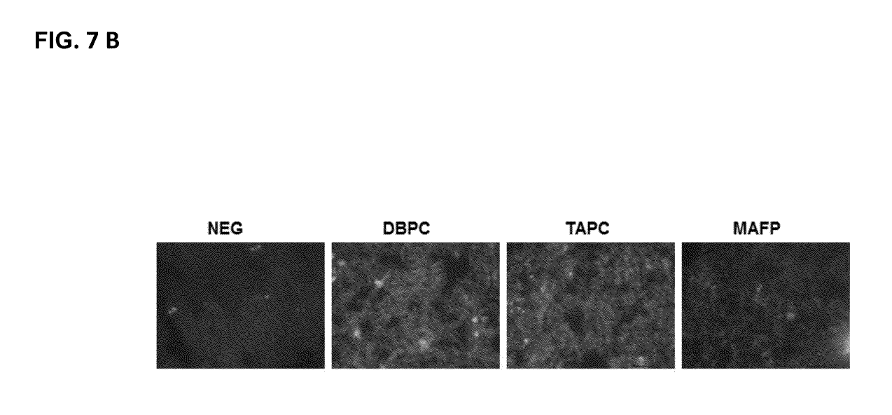

For tissue PLA₂ activities, both frozen tissue sections (in situ detection) and tissue homogenates (microplate assay) were tested. The former has the advantage of preserving tissue morphology and the cellular location of the PLA₂ activity but it was relatively difficult to quantify. Referring now FIG. 7, representative fluorescent images of PLA₂ activities in frozen tissue sections tissues from normal ovaries, benign gynecologic tumors, and ovarian cancer tissues are shown in. The total PLA₂ activities in EOC tissues (881±395 fluorescence intensity units/cell) were significantly increased compared to those in either benign (276±58) or normal (55±16) tissues (n=6 samples from each group). High levels of cytosolic PLA₂ activity throughout the section was observed in the ovarian and endometrial tumor specimens (the representative data are shown in FIG. 7A). For benign tumors, the activity was limited to the superficial cell layers (FIG. 7A). In contrast, very little or no cytosolic PLA₂ activity was observed in normal ovarian tissues (FIG. 7A). In addition, tumor PLA₂ activity was sensitive to a dual inhibitor of cPLA₂ and iPLA2b (MAFP), but not to an inhibitor of sPLA₂ (TAPC) (FIG. 7B), suggesting that the majority if not all total PLA₂ activity in tumor tissues was from cPLA₂ and/or iPLA₂.

For more quantitative PLA₂ assays, tissue homogenates (FIGS. 6B-D) were used. As in the tissue sections, the natural PLA₂ activity was elevated in EOC tumor tissue homogenates (FIG. 6B) and the effects of inhibitors support that the activities were consistent with iPLA₂ and cPLA₂ (FIG. 6C). ATX activity was also significantly different among normal/benign vs. malignant EOC tissues (FIG. 6D). Taken together, those studies suggest that EOC tissue has elevated PLA₂ and ATX activities when compared to benign gynecological and normal tissues.

### Example 8

### S1, S3 and S4, But not P3, Ascites Fractions Stimulated Proliferation of EOC Cells

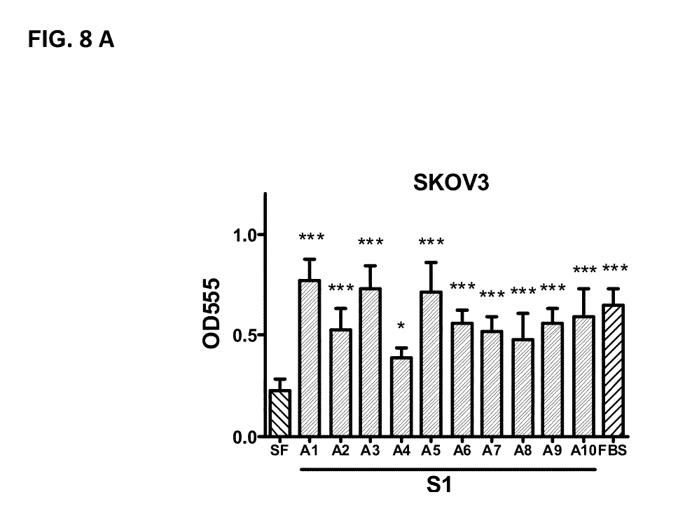

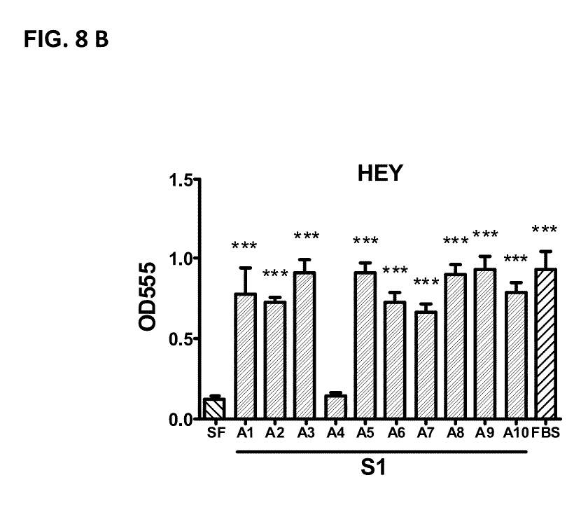

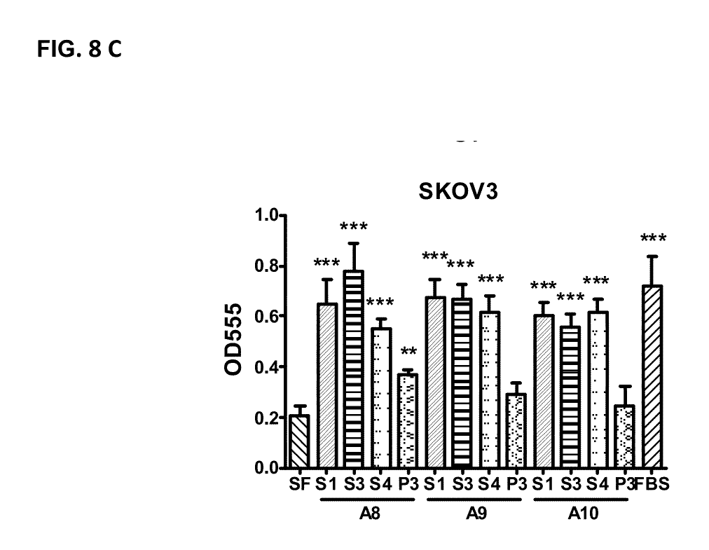

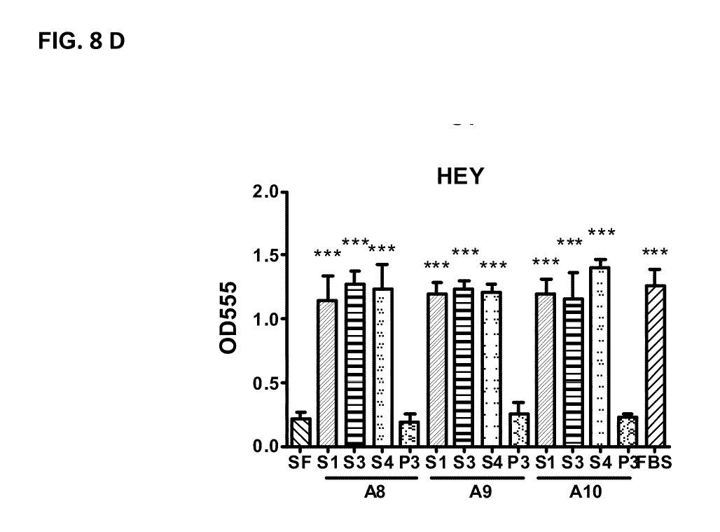

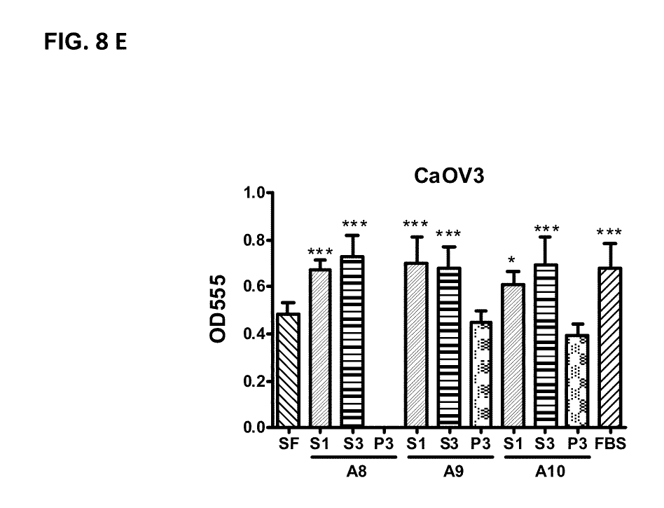

It has been shown the proliferation-stimulating activity of human EOC ascites on EOC cells more than 20 years ago. Here a set of 10 EOC ascites specimens, fractionated as described, were tested to measure their effects on proliferation in EOC cells. SKOV3 and HEY cells demonstrated a dose-responsive increase in proliferation over the range of 0.1 to 10% S1 in serum-free (SF) medium, with maximum effect at 5% 51 (detailed data not shown). Referring now to FIG. 8A, cells in 96-well plates were treated with 5% 51, S3, S4 ascites fractions in serum-free medium (SF) or an amount of the P3 fraction equal to 2.5× the concentration in the original ascites. SF medium and SF containing 5% FBS were used as controls.

After 72 hr incubation, the MTT assay was conducted and absorbance at 555 nm recorded. All 10 S1 fractions (at 5%) stimulated proliferation with similar or higher potency as compared to FBS (5%) in SKOV3 cells (FIG. 8A). These effects were similar in HEY cells, except that one sample had low activity (FIG. 8B). The S3 fraction (at 5%) from different ascites specimens had almost identical proliferation-stimulating activity when compared to S1 in all three EOC cells lines tested (SKOV3, HEY and CaOV3; FIGS. 8C-E), while the P3 fraction had much lower or no activity. The effect of ascites from 3 patients and respective fractions on proliferation of SKOV3, HEY, and CaOV3 cells, were *P<0.05; **P<0.01; ***P<0.001 compared to SF medium. Data are mean and SD of 4-6 replicates. Experiments were repeated at least twice. The S4 fraction (at 5%) had very similar activity as S1 and S3, suggesting the majority of human EOC ascites proliferation-stimulating activity is not associated with exosomes. Proliferation of SKOV3 cells was reduced by 28.8±9.98% (P<0.001) by 100 μM MAFP, by 24.6±13.2% by 25 μM BrP-LPA (P=0.006), and by 12.4±7.44% (P=0.016) by 10 μM Ki16425, showing involvement of PLA₂ and ATX enzymes and LPA receptors, respectively, in the stimulation of proliferation by S1.

### Example 9

### S1, S3 and S4, But not P3, Ascites Fractions Potently Stimulated Migration and Invasion of EOC Cells

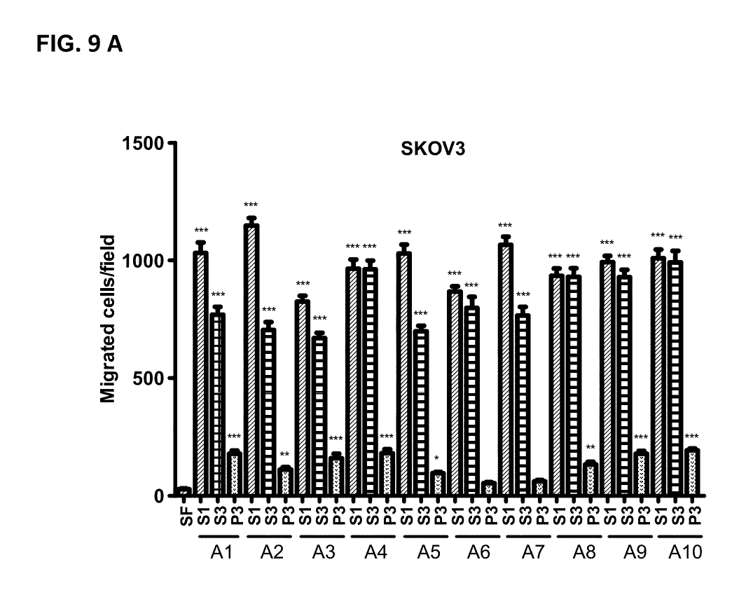

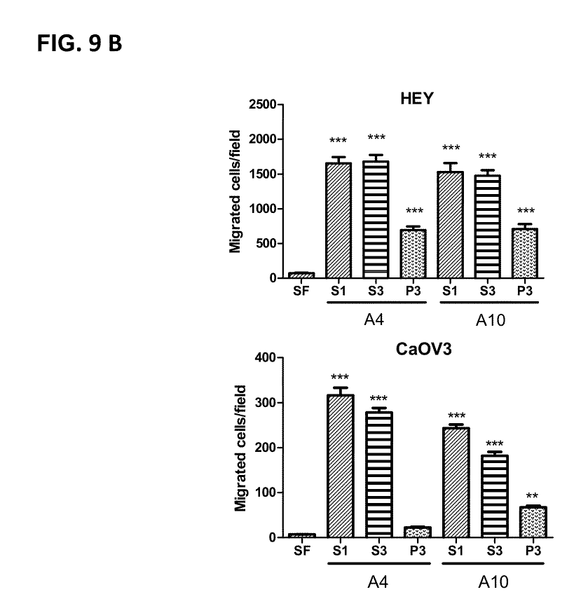

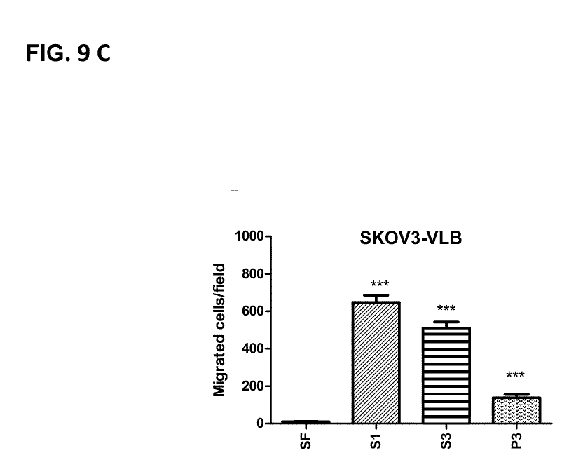

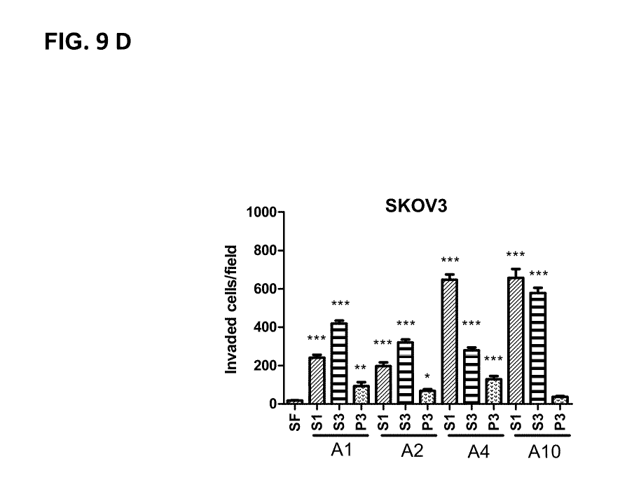

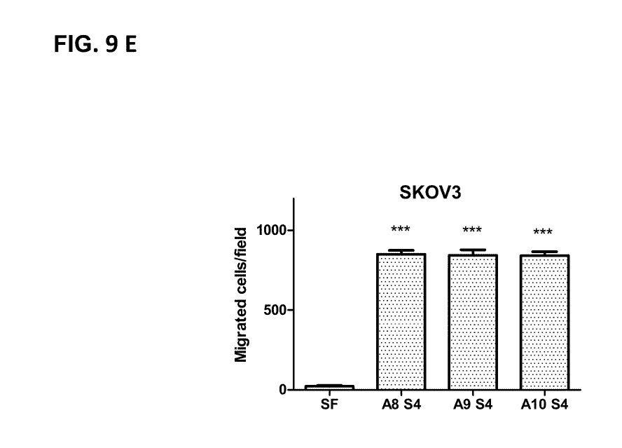

Human S1 and S3 ascites fractions dose-dependently stimulated migration of SKOV3 cells, reaching a maximum effect at 1% ascites in SF medium (not shown). All 10 S1 and S3 ascites fractions potently stimulated migration of SKOV3 cells, with the S3 fraction having approximately 60-90% of the activity of the S1 fraction. In contrast, the P3 fraction had only about 10% of the activity of S1 (FIG. 9A). Migration of HEY and CaOV3 cells was also stimulated by S1, S3, and P3 ascites fractions (FIG. 9B). In HEY and CaOV3 cells, S3 fractions had 80-100% of the activity of S1, and P3 had 0-30% of the activity of S1 in stimulating cell migration. In addition, the migration-stimulating effects of ascites fractions on the drug-resistant cell line SKOV3-VLB was very similar to the effects seen in SKOV3 cells, showing that the stimulatory effect of ascites was maintained in this multi-drug resistant cell line (FIG. 9C). S1, S3, and P3 fractions also stimulated cell invasion (FIG. 9D), with the S4 fraction having very similar activity as compared to S3 (FIG. 9E).

### Example 10

### LPA is a Major Mediator of the Tumor-Promoting Activity of Ascites Fractions

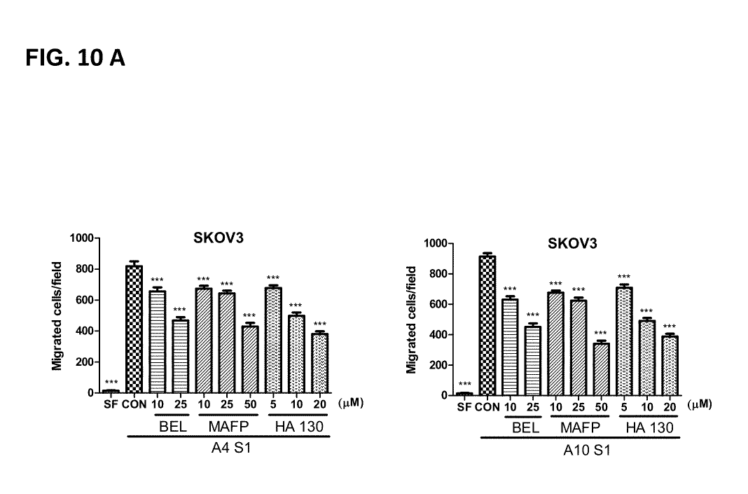

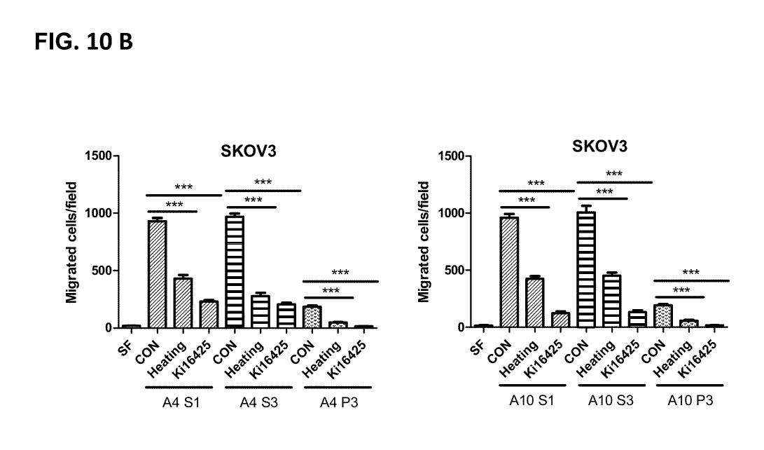

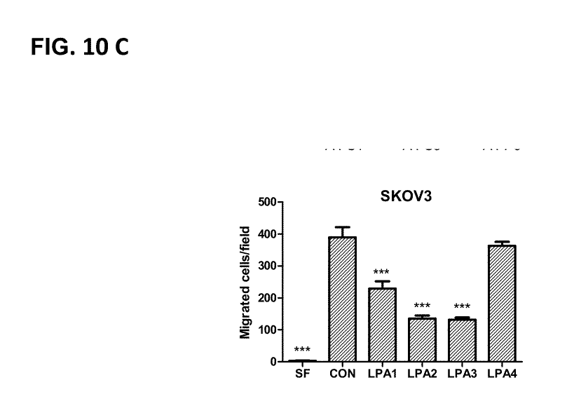

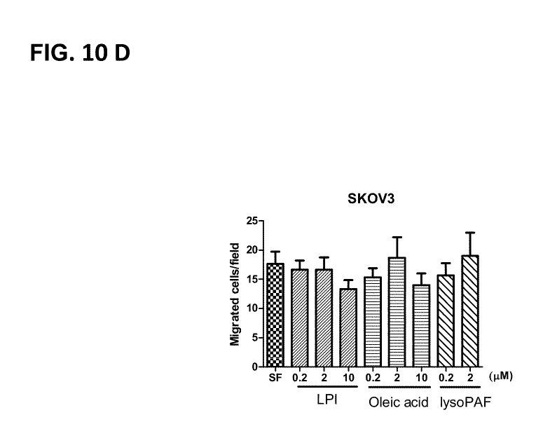

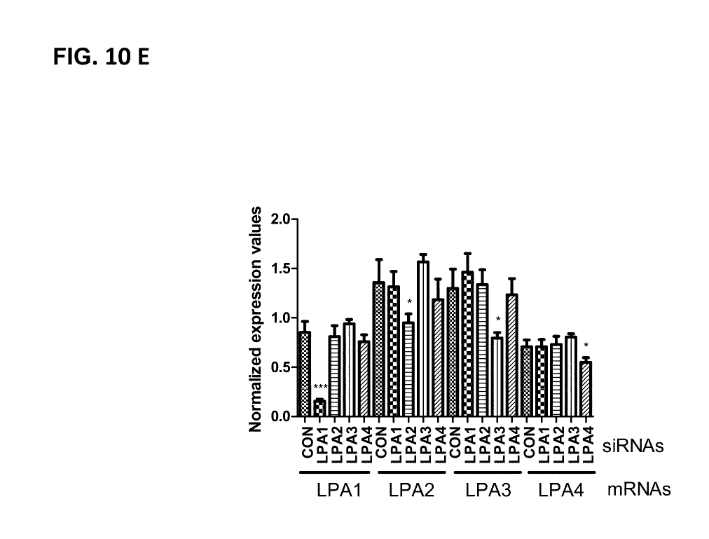

In order to determine whether PLA₂ and ATX were functionally involved in ascites-induced migration of EOC cells, the inhibitors MAFP, BEL, and HA130 were added to the lower chambers during the assay. All three inhibitors dose-dependently reduced migration of SKOV3 cells induced by two different S1 fractions (FIG. 10A). The effect of each inhibitor was almost identical whether using S1, S3, or S4 fractions (FIG. 10 and data not shown).

While the effect of LPA on EOC cell migration and invasion, has been reported, it remained to be tested whether LPA was a component of the effects of S1 and S3 ascites fractions. First, S1 and S3 fractions were treated at 95° C. for 10 min and then their effect on migration was measured (FIG. 10B). Approximately 40 and 50% of the migration-stimulating activity was retained using SKOV3 (FIG. 10B) and HEY cells (data not shown), respectively, suggesting that a significant portion of the activity is likely mediated by heat-resistant lipid factors. Second, Ki16425, a selective blocker of LPA₁ and LPA₃ receptors, was used and it was found that it inhibited >70% of the migratory activity induced by 51, S3, and P3 fractions (FIG. 10B). Third, specific siRNAs for LPA₁₋₄ was used.

Referring now to FIG. 10 A (left panel), values were also measured in the presence of 2 different concentrations of BEL, MAFP, or HA 130. T LPA mediated the tumor-promoting activity of ascites fractions. The effect of PLA₂ and ATX inhibitors on ascites S1-induced migration. ***P<0.001 compared to control (CON). These siRNAs effectively and specifically down-regulated each of these receptors at the mRNA level (FIG. 10E). siRNAs against LPA₁₋₃, but not LPA₄, significantly reduced S1-induced cell migration (FIG. 10C).

It has been shown that one of the major PLA₂ products, LPC, does not stimulate EOC cell migration. Lysophosphatidylinositol (LPI), another major lysophospholipid (LPL) that is elevated in human EOC ascites, oleic acid, and lyso-platelet activating factor (lyso-PAF) was tested, and it was found that they did not stimulate migration or invasion (FIG. 10D). Collectively, those data support that LPA, present and produced in EOC ascites fractions, is a major mediator of the migration-promoting effects. All 10 S1 and S3 specimens stimulated proliferation (1.4-2.6 fold), migration (3-40 fold), and invasion (4-35 fold) of EOC cells. These functions were sensitive to MAFP (15-40% inhibition), BEL (20-40%), and the ATX inhibitor HA 130 (10-45% inhibition), supporting the involvement of PLA₂ and ATX activities in EOC cell migration.

### Example 12

### Ex Vivo Lipid Generation in S1 and S4 Ascites Fractions is Time-Dependent and Sensitive to MAFP, BEL, and BrP-LPA

Active enzymatic lipid generation in EOC ascites were examined using the previously developed MS-based lipidomics approach. Lipid levels in S1 and S4 ascites fractions incubated at 37° C. at zero and 48-hr time points were measured. Incubation of S1 ascites fractions (n=3) produced a 6-9-fold increase in total LPA concentration over 48 hr, as well as increases in other LPLs and fatty acids (FA), including AA (3-5-fold increase; Table 4). In addition, lipid generation was highly sensitive to MAFP (74-80% inhibition) and much less sensitive to BrP-LPA (13-28% inhibition) (Table 4). The S1 and S4 fractions had very similar patterns of lipid generation (Table 4). In contrast, P3 did not generate LPA under similar conditions (not shown), indicating that the majority, if not all, of the LPA-producing activity in ascites is soluble and not associated with vesicles (including exosomes).

TABLE 4
Lipid generation in ascites samples ex vivo. S1 and S4 ascites fractions were incubated
at 37° C. for 48 hrs in the presence or absence of inhibitors (100 μM).
Lipids were analyzed by LC-MS and are expressed as μM concentration in ascites.

| ID, | | total | | total | ID, | | total | total |
|---|---|---|---|---|---|---|---|---|
| fraction | Condition | LPC | AA | LPA | fraction | Condition | LPC | LPA |
| A5 S1 | 0 hr | 73.4 | 1.1 | 7.5 | A5 S4 | 0 hr | 68.5 | 8.0 |
| | 48 hr | 49.3 | 3.2 | 43.2 | | 48 hr | 45.7 | 44.6 |
| | MAFP | 65.8 | 3.1 | 14.2 | | MAFP | 64.6 | 14.2 |
| | BrP-LPA | 63.7 | 2.8 | 34.4 | | BrP-LPA | 64.3 | 33.5 |
| A6 S1 | 0 hr | 51.3 | 1.9 | 4.3 | A6 S4 | 0 hr | 56.4 | 5.8 |
| | 48 hr | 49.1 | 6.7 | 34.9 | | 48 hr | 41.7 | 36.8 |
| | MAFP | 55.3 | 5.4 | 6.9 | | MAFP | 54.8 | 12.3 |
| | BrP-LPA | 57.2 | 4.8 | 24.5 | | BrP-LPA | 58.6 | 29.1 |
| A7 S1 | 0 hr | 39.2 | 2.1 | 4.9 | A7 S4 | 0 hr | 44.6 | 6.8 |
| | 48 hr | 28.2 | 5.4 | 42.5 | | 48 hr | 31.8 | 47.3 |
| | MAFP | 46.2 | 4.3 | 12.0 | | MAFP | 49.4 | 16.1 |
| | BrP-LPA | 56.2 | 4.8 | 39.2 | | BrP-LPA | 58.3 | 45.1 |

Further analysis of changes in individual LPC and LPA species support the role of PLA₂ in LPA production (Table 5). During the incubation, the levels of LPC, the product of PLA₂ and the substrate of ATX, decreased, consistent with the conversion of LPC to LPA by the ascites ATX activity. However, it is interesting to note that in the S1 fractions of three individual ascites tested after 48 hr incubation, the total LPC levels decreased by 24, 2, and 11 μM and the corresponding total LPA levels increased by 36, 30, and 38 μM.

TABLE 5
Individual LPC and LPA lipid species in control and incubated ascites fractions from three EOC patients.
S1 and S4 ascites fractions were incubated at 37° C. for 48 hrs and lipids were analyzed by LC-MS.
Lipids are expressed as μM concentration in ascites. Inhibitors were added for the 48 hr incubation (100 μM).

| ID and | | | 16-0 | 18:2 | 18:1 | 18:0 | 20:4 | 22:6 | 16:0 | 18:2 | 18:1 | 18:0 | 20:4 | 22:6 |
|---|---|---|---|---|---|---|---|---|---|---|---|---|---|---|
| Fraction | | Condition | LPC | LPC | LPC | LPC | LPC | LPC | LPA | LPA | LPA | LPA | LPA | LPA |
| A5 | S1 | Control | 43.9 | 7.05 | 8.96 | 9.80 | 2.34 | 0.77 | 2.10 | 1.77 | 0.90 | 0.30 | 2.04 | 0.31 |
| | | 37° C., 48 hr | 31.7 | 2.37 | 4.51 | 8.88 | 1.03 | 0.48 | 18.1 | 6.72 | 6.25 | 2.60 | 7.62 | 1.59 |
| | | 37° C., 48 hr + | 39.3 | 5.82 | 7.90 | 9.13 | 2.13 | 0.89 | 3.88 | 3.46 | 1.72 | 0.61 | 3.83 | 0.63 |
| | | MAFP | | | | | | | | | | | | |
| | | 37° C., 48 hr + | 39.8 | 3.58 | 6.84 | 10.9 | 1.50 | 0.78 | 13.2 | 6.39 | 4.55 | 1.95 | 6.76 | 1.27 |
| | | BrP-LPA | | | | | | | | | | | | |
| A5 | S4 | Control | 41.4 | 6.04 | 8.37 | 9.30 | 2.21 | 0.66 | 2.20 | 1.99 | 0.94 | 0.34 | 2.15 | 0.34 |
| | | 37° C., 48 hr | 28.9 | 2.52 | 4.55 | 7.96 | 1.05 | 0.44 | 17.5 | 7.49 | 6.68 | 2.71 | 8.13 | 1.69 |
| | | 37° C., 48 hr + | 38.2 | 5.96 | 7.91 | 8.85 | 2.28 | 0.90 | 3.95 | 3.49 | 1.79 | 0.61 | 3.57 | 0.61 |
| | | MAFP | | | | | | | | | | | | |
| | | 37° C., 48 hr + | 38.5 | 3.95 | 7.79 | 11.1 | 1.80 | 0.81 | 12.8 | 6.26 | 4.51 | 1.89 | 6.61 | 1.17 |
| | | BrP-LPA | | | | | | | | | | | | |
| A6 | S1 | Control | 33.0 | 2.37 | 5.40 | 6.96 | 2.67 | 0.58 | 1.13 | 0.50 | 0.39 | 0.20 | 1.80 | 0.22 |
| | | 37° C., 48 hr | 32.7 | 1.44 | 3.99 | 9.00 | 1.28 | 0.39 | 14.9 | 2.85 | 4.61 | 2.23 | 8.76 | 1.33 |
| | | 37° C., 48 hr + | 33.7 | 3.17 | 6.15 | 7.69 | 3.27 | 0.84 | 1.70 | 0.83 | 0.66 | 0.31 | 2.94 | 0.34 |
| | | MAFP | | | | | | | | | | | | |
| | | 37° C., 48 hr + | 36.1 | 2.12 | 5.99 | 9.60 | 2.33 | 0.70 | 9.65 | 2.36 | 2.89 | 1.38 | 7.11 | 0.90 |
| | | BrP-LPA | | | | | | | | | | | | |
| A6 | S4 | Control | 35.8 | 2.98 | 5.92 | 7.47 | 3.13 | 0.66 | 1.72 | 0.62 | 0.59 | 0.26 | 2.32 | 0.26 |
| | | 37° C., 48 hr | 26.9 | 1.62 | 3.62 | 7.27 | 1.61 | 0.46 | 14.3 | 3.34 | 4.49 | 2.21 | 10.70 | 1.44 |
| | | 37° C., 48 hr + | 33.7 | 3.23 | 6.12 | 7.52 | 3.18 | 0.73 | | | | | | |
| | | MAFP | | | | | | | | | | | | |
| | | 37° C., 48 hr + | 35.0 | 2.36 | 6.64 | 10.5 | 2.87 | 0.88 | 10.3 | 3.10 | 3.08 | 1.57 | 9.73 | 1.07 |
| | | BrP-LPA | | | | | | | | | | | | |
| A7 | S1 | Control | 20.8 | 4.45 | 5.36 | 4.95 | 2.88 | 0.48 | 0.70 | 1.07 | 0.48 | 0.20 | 2.21 | 0.25 |
| | | 37° C., 48 hr | 16.9 | 1.51 | 2.34 | 6.03 | 1.04 | 0.23 | 16.2 | 4.98 | 6.17 | 2.85 | 10.54 | 1.49 |
| | | 37° C., 48 hr + | 26.1 | 4.19 | 5.89 | 6.52 | 2.60 | 0.57 | 2.00 | 2.40 | 1.31 | 0.48 | 5.06 | 0.61 |
| | | MAFP | | | | | | | | | | | | |
| | | 37° C., 48 hr + | 33.5 | 2.62 | 6.17 | 11.2 | 1.91 | 0.50 | 13.3 | 5.22 | 5.61 | 2.50 | 11.01 | 1.33 |
| | | BrP-LPA | | | | | | | | | | | | |
| A7 | S4 | Control | 24.0 | 4.50 | 6.11 | 6.26 | 3.04 | 0.48 | 1.25 | 1.38 | 0.72 | 0.29 | 2.82 | 0.30 |
| | | 37° C., 48 hr | 18.7 | 1.78 | 2.61 | 7.10 | 1.13 | 0.25 | 17.3 | 5.84 | 7.25 | 3.01 | 11.82 | 1.65 |
| | | 37° C., 48 hr + | 27.8 | 4.62 | 6.30 | 6.98 | 2.78 | 0.60 | 2.95 | 3.24 | 1.83 | 0.66 | 6.57 | 0.72 |
| | | MAFP | | | | | | | | | | | | |
| | | 37° C., 48 hr + | 34.2 | 2.91 | 6.47 | 11.8 | 2.08 | 0.56 | 15.9 | 6.16 | 6.35 | 2.92 | 12.09 | 1.29 |
| | | BrP-LPA | | | | | | | | | | | | |

Where the yield of LPA was higher than the loss of LPC, a reasonable assumption is that PLA₂ was actively providing LPC substrate for ATX (Table 4). This notion was further supported by a closer look at the individual LPC and LPA species (Table 5). For the 16:0, 18:2, and 18:1 species, the decreased amounts of LPC correlated well with concomitant increase in LPA, suggesting a near stoichiometric conversion of LPC to LPA by ATX. However, for other species (18:0, 20:4, and 22:6), the molar increases in LPAs were 2-5 fold higher than the decreases in corresponding LPCs, suggesting the action of PLA₂s in regenerating these LPC species (from PC) which were actively being hydrolyzed by ATX. The presence of phosphatidic acid (PA) species detected in ascitic samples (1.5-7 μM) also implying the possibility that PLA₂s may use these substrates in generating LPAs directly. Following ex vivo incubation, the 48 hr changes in LPC and LPA species when comparing S1 and S4 fractions were strikingly similar (correlation coefficient r=0.936, n=72, P<0.0001), validating the methodology and further supporting that PLA₂ activity is associated with a vesicle-free fraction of ascites.

### Example 13

### Effect of MAFP in EOC Models In Vivo

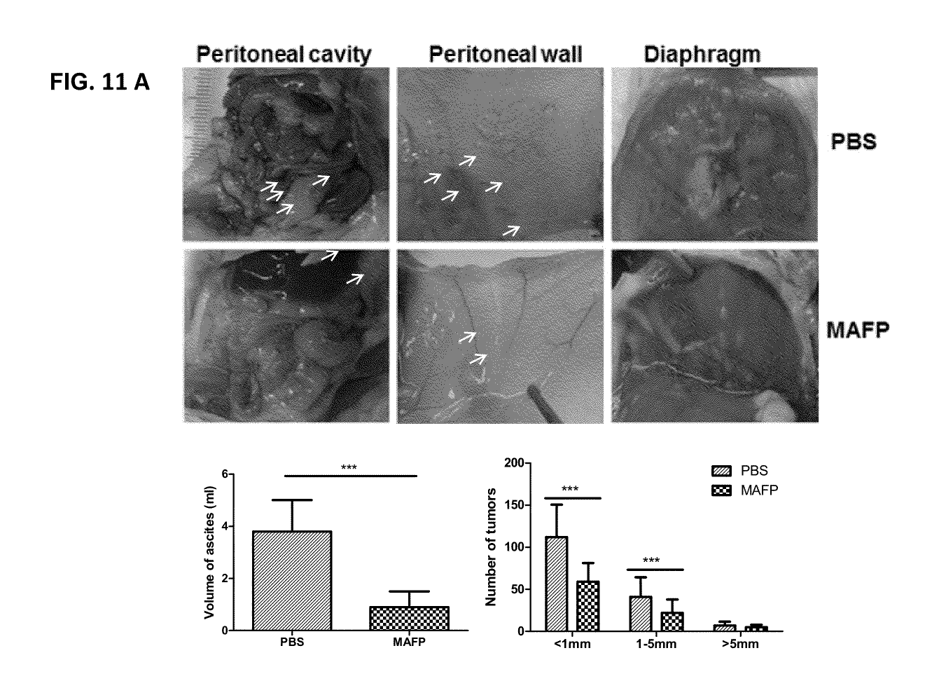

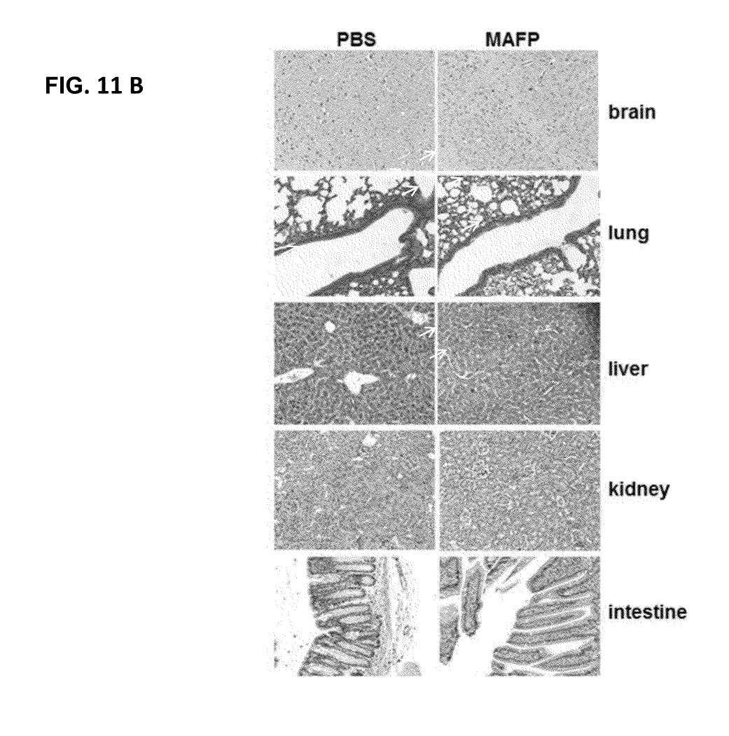

As shown previously, the inhibitors BEL and iPLA₂ have a combinational effect when used with low and non-toxic doses of paclitaxel; used together these compounds can inhibit EOC metastasis in the mouse model for this disease. The data reported herein suggest that MAFP is more effective than BEL in blocking PLA₂ activity and migration/invasion-inducing activity of ascites S1 and S3 fractions. MAFP was tested to determine if it would be effective in the mouse model of EOC used herein. NOD/SCID mice were injected i.p. with SKOV3-Luc cells; treatment with MAFP or vehicle was initiated 10 days later. Referring now to FIG. 11. Human SKOV3 cells were injected i.p. into NOD/SCID mice. Ten days later, i.p. injection of MAFP or PBS was initiated. Mice were sacrificed at 38-40 days. MAFP treatment decreased tumor formation in a mouse xenograft model of EOC. Tumor numbers and sizes were significantly reduced in the MAFP-treated versus the vehicle-treated group (FIG. 11A). Additionally, MAFP caused no detectable toxicity in the major tissues assayed, including the brain, lung, liver, kidney, and intestine (FIG. 11B).

Two additional mouse studies were performed to confirm that soluble factors in S4 (and not the exosome fraction P3) were tumor-promoting in vivo. In one study, HEY ovarian cancer cells, which have low tumorigenicity (36), were tested to determine if they would be more tumorigenic in the presence of S4. Beginning 2 days after i.p. tumor cell injection, 0.5 mL pooled S4 or PBS was injected i.p. 3 times/wk. At 3 wks, all mice in the S4 group (n=7) had many tumors throughout the peritoneum, while in the PBS group (n=5) only one mouse had many tumors; one mouse had few tumors and 3 mice had a small tumor only at the injection site. In another study, SKOV3-Luc cells i.p. were injected into the mice and 10 days later an injection of a pooled P3 fraction (containing 500 μg protein) (n=3) or PBS (n=3) was started. This treatment was continued 2 times/wk for 3 wks. There was no difference in tumor or ascites development between the two groups.

### Example 14

### EOC Cells or Macrophages Secrete cPLA₂ and iPLA₂ in ABC-Transporter-Dependent, but Exosome Secretion-Independent Manner

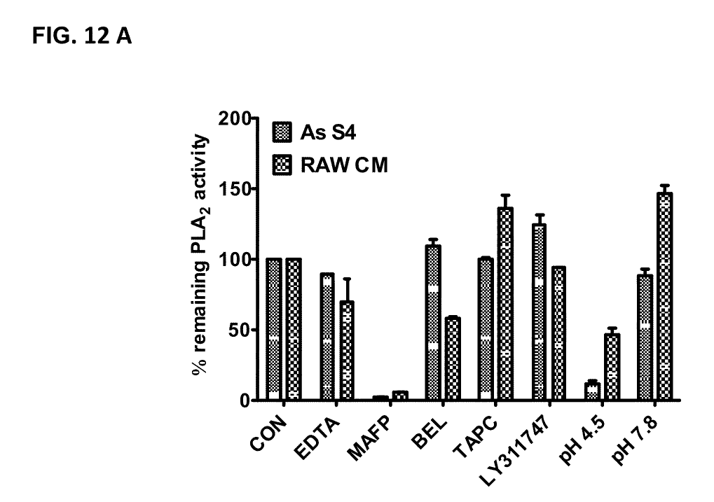

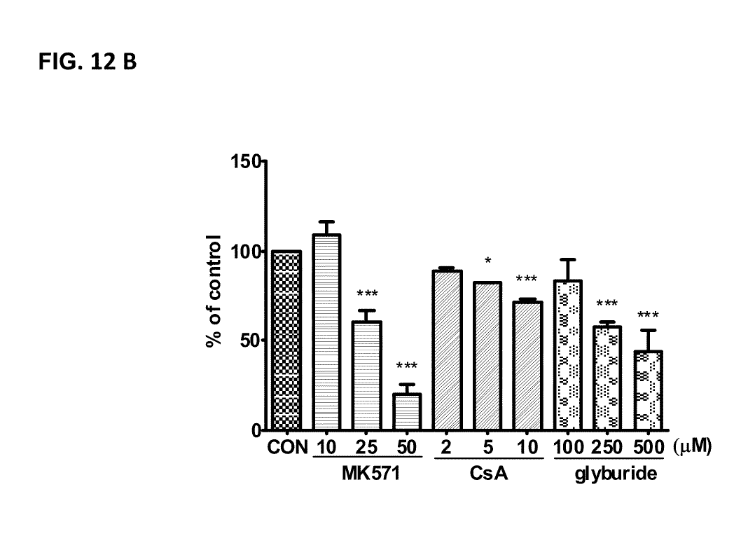

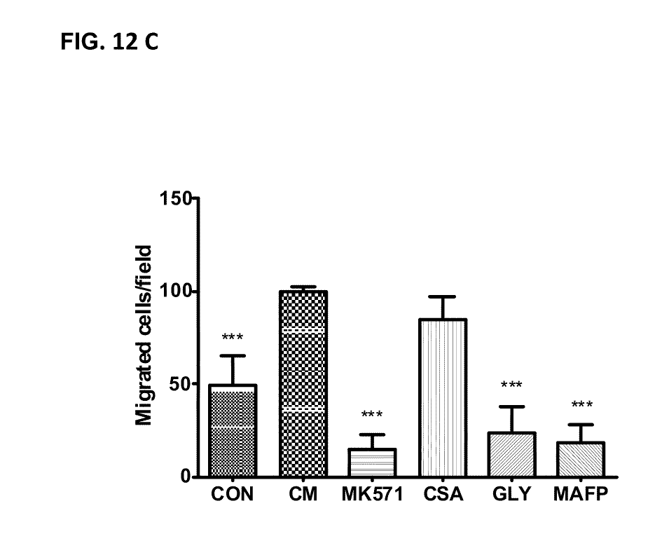

In order to understand the mechanisms of PLA₂ secretion from RAW 264.7 mouse macrophages were tested using two inhibitors known to inhibit exosome secretion: 5,5-(N—N-Dimethyl)-amiloride hydrochloride (DMA) (5 and 12.5 nM) and methyl-β-cyclodextrin (MBC), a lipid-raft pathway inhibitor which reduces exosome secretion (1 and 2 mM) (37). Neither inhibitor had any significant effect on the PLA₂ activity detected in CM of RAW 264.7 mouse macrophages (not shown). Referring now to FIG. 12B. The sensitivity of PLA₂ activity of conditioned medium (CM) from RAW 246.7 macrophages to inhibitors, EDTA or pH was compared to that of ascites S4 fraction. Inhibitors concentrations were EDTA (5 mM), MAFP (10 μM), BEL (10 μM), TAPC (50 μM), and LY311747 (50 μM). These data show that conditioned medium from RAW 246.7 macrophages contains PLA₂ and cell stimulatory activity. This activity was sensitive to PLA₂ inhibitors in a manner that was similar to the EOC ascites fractions (FIG. 12A), suggesting similar PLA₂ enzymes were present. The assays were also run at a very low pH (4.5) and a higher pH (7.8) to test whether lysosomal PLA₂ (LPLA₂) activity, which is maximal at low pH, was present. The data indicate that LPLA₂ activity is not significant in ascites S4 or in RAW 264.7 macrophage CM.

Referring now to FIG. 12B, the PLA₂ activity of 17-hr CM from cells treated for 4 hr with inhibitors of ABC transporters, normalized to cell protein and expressed as % of untreated cells. *P<0.05; ***P<0.001 compared to control (CON, medium alone). Data are mean and SD of duplicate treatments, experiment repeated three times with similar results. CM was collected from RAW macrophages treated with selective inhibitors of different ATP-binding cassette (ABC) transporters: MK571, an ABCC1 inhibitor (10-50 μM); cyclosporine A (CsA), an ABCB1 inhibitor (2-50 μM); and glyburide (Gly), an ABCA1 inhibitor (0.1-2 mM) (38). RAW CM PLA₂ activities, normalized to μg of cell protein, are presented in FIG. 12B. All inhibitors dose-responsively reduced PLA₂ activity of CM with the order of effectiveness being MK571>glyburide>CsA. These results are consistent with the findings that human ascitic PLA₂ activity was not associated with P3 (the exosome-containing fraction), supporting the idea that PLA₂s are secreted in a vesicle-independent pathway.

Referring now to FIG. 12C, CM from RAW 264.7 mouse macrophages was tested to determine if it would stimulate migration of SKOV3 cells. Migration of SKOV3 cells to 17-hr CM from untreated cells and cells treated with inhibitors (MK571, 50 μM, CsA, 10 μM, glyburide, 500 μM, and MAFP, 20 μM). ***, P<0.001 compared to CM. Data is mean and SD of three membranes; experiment repeated twice. An approximately 2-fold increase in migration with the CM compared to the medium control in the bottom well of the migration chamber was found. CM generated from RAW 264.7 macrophages treated with MK571 or glyburide did not stimulate migration. The addition of MAFP to the CM also abrogated migration. These results further support a PLA₂-ABC transporter system operating in the tumor ascites microenvironment.

The findings disclosed herein unexpectedly indicate that the cytosolic enzymes cPLA₂ and iPLA₂ are associated with microvesicle-free biological fluids. If the enzymes originate in live cells, they could come from tumor cells, immune system cells, peritoneal mesothelial cells, and/or the interaction of two or more cell types. The release of cPLA₂- and/or iPLA₂-like activity in conditioned medium (CM) from EOC cell lines SKOV3 and HEY were tested for. A small amount of activity but not enough to account for the PLA₂ activity of EOC ascites (data not shown) was found. In CM from RAW 264.7 mouse macrophages, however, a high level of PLA₂ activity was found.

### Materials and Methods

### Human Sample Collection and Processing.

Human Ascites and tissue samples were obtained from Department of OB/GYN, Indiana University School of Medicine (IUSM) and Cleveland Clinic or through Cooperative Human Tissue Network (CHTN), a NIH sponsored organization providing human tissues to researchers under approved Institutional Review Board (IRB) protocols. Ascites from EOC patients were kept at 4° C. throughout processing and fractions were aliquoted and stored at −80° C. Samples were centrifuged on the day of collection at 3000×g for 20 min to sediment cells and debris. The supernatant (51) was further fractionated by centrifuging at 20,000×g for 20 min, resulting in S2 and pellet 2 (P2, cell fragments and large vesicles). S2 was ultra-centrifuged at 110,000×g for 2 hr, resulting in S3 and P3 (exosomes). A final centrifugation of S3 at 200,000×g for 2 hr resulted in S4 and P4 (other microvesicles). P3 and P4 fractions were resuspended in cold PBS and subjected to another ultracentrifugation before final suspension in PBS. Snap-frozen tissues were collected from surgically removed malignant (from both primary and metastatic sites) ovarian tumors or benign tumors, along with adjacent normal tissues for both.

The demographic data for human ascites and tissue samples are shown in Tables 1 and 2.

TABLE 1
Demographic data for human ascites samples.

| | | | | | | Previous |
|---|---|---|---|---|---|---|
| ID | Age | Race | Histopathology | Grade | Stage | chemo. |
| A1 | 70 | Caucasian | Papillary serous | 3 | III-C | Yes |
| | | | adenocarcinoma | | | |
| A2 | 73 | Caucasian | Papillary serous | 3 | IIIC | Yes |
| | | | adenocarcinoma, | | | |
| | | | clear cell carcinoma | | | |
| A3 | 51 | Caucasian | Papillary serous | 3 | IV | Yes |
| | | | adenocarcinoma | | | |
| A4 | 66 | Caucasian | Peritoneal | Not | IV | Yes |
| | | | carcinomatosis | Graded | | |
| A5 | 50 | Caucasian | Papillary serous | 3 | IIIC | Yes |
| | | | adenocarcinoma | | | |
| A6 | 63 | Caucasian | Endometroid | 2 | III | Yes |
| | | | adenocarcinoma | | | |
| A7 | 54 | Caucasian | Papillary serous | 3 | IIIC | No |
| | | | adenocarcinoma | | | |
| A8 | 65 | Caucasian | Papillary serous | 3 | IIC | Yes |
| | | | adenocarcinoma | | | |
| A9 | 47 | Caucasian | Mixed endometrioid and | 1 | IIIC | No |
| | | | mucinous adenocarcinoma | | | |
| A10 | 72 | African | Papillary serous | Not | IV | No |
| | | American | adenocarcinoma | Graded | | |

TABLE 2
Demographic data for human tissue samples.

| ID | Age | Race | Histopathology | Stage | Grade |
|---|---|---|---|---|---|
| N1 | 49 | Caucasian | Normal ovary | | |
| N2 | 46 | African American | Normal ovary | | |
| N3 | 51 | Caucasian | Normal ovary | | |
| N4 | 62 | Caucasian | Normal ovary | | |
| N5 | 47 | Caucasian | Normal ovary | | |
| N6 | 59 | Caucasian | Normal ovary | | |
| N7 | 69 | African American | Normal ovary | | |
| N8 | 54 | Caucasian | Normal ovary | | |
| N9 | 50 | Caucasian | Normal ovary | | |
| N10 | 45 | Caucasian | Normal ovary | | |
| B1 | 44 | Caucasian | Benign ovary | | |
| B2 | 80 | Caucasian | Benign ovarian | | |
| | | | cystadenoma | | |
| B3 | 49 | Caucasian | Benign ovarian | | |
| | | | cystadenoma | | |
| B4 | 42 | Caucasian | Benign ovarian | | |
| | | | fibroma | | |
| B5 | 27 | Other | Benign ovary | | |
| B6 | 34 | Caucasian | Benign ovary | | |
| B7 | 36 | Caucasian | Benign ovary | | |
| B8 | 37 | Caucasian | Benign ovary | | |
| B9 | 42 | Caucasian | Benign ovary | | |
| B10 | 31 | Caucasian | Benign ovary | | |
| O1 | 76 | Caucasian | Serous | II | III |
| | | | adenocarcinoma | | |
| O2 | 55 | Caucasian | Mucinous | I | I |
| | | | adenocarcinoma | | |
| O3 | 70 | Caucasian | Papillary serous | II | III |
| | | | adenocarcinoma | | |

### Reagents and Inhibitors.

The PLA₂ substrate 1-O-(6-Dabcyl-Aminohexanoyl)-2-O-(6-(12-BODIPY-Dodecanoyl) Aminohexanoyl)-sn-3-Glyceryl Phosphatidylcholine (DBPC) and the ATX substrate FS-3 were from Echelon Bioscience (Salt Lake City, Utah, USA). The radio-labeled substrate 1-palmitoyl-2-[1-¹⁴C] palmitoyl-sn-glycero-3-phosphocholine was from PerkinElmer (Boston, Mass., USA). siRNAs were from Santa Cruz Biotechnology (Santa Cruz, Calif., USA). BEL and methyl arachidonyl fluorophosphonate (MAFP) were from Santa Cruz Biotechnology (Santa Cruz, Calif., USA); arachidonyl trifluoromethyl ketone (AACOCF3) was from EMD Chemicals (Philadelphia, Pa., USA); thioetheramide-PC (TAPC) was from Cayman Chemical Co. (Ann Arbor, Mich., USA); and BrP-LPA was from Echelon Bioscience (Salt Lake City, Utah, USA). Antibody to cPLA₂ was from Cell Signaling (Danvers, Mass., USA); antibody to iPLA₂ was from Cayman Chemical (Ann Arbor, Mich., USA).

### PLA₂ Enzymatic Activity Analyses.

To prepare tissue homogenate, pulverized tissue (100-150 mg) was mixed with 500 μL lysis buffer (10 mM Hepes, 0.34M sucrose, pH 7.5) containing protease inhibitor cocktail (Sigma-Aldrich, St Louis, Mich., USA). Samples were homogenized with an Omni Tissue Homogenizer (Kennesaw, Ga., USA) and centrifuged at 16,000×g at 4° C. for 40 min. The supernatant was removed to a fresh tube and a small aliquot was retained for protein assay (BCA assay, Thermo-Fisher Scientific, Rockford, Ill., USA).

PLA₂ activities were analyzed using the fluorescent substrate 1-O-(6-Dabcyl-aminohexanoyl)-2-O-(12-(5-BODIPY-pentanoyl)aminodecanoyl)-sn-3-glyceryl phosphatidylcholine (DBPC), a fluorogenic phosphatidylcholine (PC) substrate (16). A radio-labeled PC substrate was also used to validate the DBPC-based method as previously described (16). For the DBPC-based assays, tissue homogenate (0.02 mg protein) or human EOC ascites (1.0 μL) were mixed with DBPC (0.2 μg in 200 μL of PBS). The PLA₂ activities were expressed as change in fluorescence intensity/min/mg of protein or /μL of body fluid.

For frozen tissue section PLA₂ assays, the snap frozen tumors were embedded in OCT and sliced into 10-μm sections on glass slides. The sections were fixed with cold methanol for 20 min, and then incubated with cold PBS for 5 min. The sections were incubated with DBPC (0.1 μg in 100 μL 1% DMSO in PBS) for 1 hr at room temperature, and then washed with PBS. Cell nuclei were co-stained by the use of VECTASHIELD Mounting Medium with DAPI (Vector Laboratories Inc., Burlingame, Calif.). Inhibitors (BEL (40 μM), MAFP (40 μM; a dual inhibitor of cPLA₂ and iPLA₂), or TAPC (250 μM) in PBS/DMSO (100/1, v/v)), were pre-incubated with the frozen sections for 5 min prior to adding DBPC. The fluorescence was imaged using a Nikon fluorescent microscope (ECLIPSE TE2000-S, Melville, N.Y.). The quantification of the activity was performed using the Show Region Statistics function in the MetaMorph software and expressed as fluorescence intensity/cell.

### PLA₂ Activity in Conditioned Medium (CM).

For detection of secreted PLA₂ activity, a near confluent monolayer of RAW 264.7 mouse macrophages was rinsed with PBS and covered with one half volume of serum-free supportive medium (DMEM/F12 with 1% ITS, 0.1% BSA and antibiotics). Samples of the CM were taken and analyzed for PLA₂ activity using the fluorescent DBPC method. To test inhibitors of cell secretion, the inhibitor was added to the cells in serum-free medium and incubated for 4 hr. Inhibitors were removed and fresh medium added. After 15-18 hr incubation, samples of CM were assayed for PLA₂ activity as described. Amiloride, 5-(N,N-dimethyl)-hydrochloride (DMA), brefeldin A (BFA), cyclosporine A (CsA), and glyburide were obtained from Santa Cruz Biotechnology (Santa Cruz, Calif., USA). Methyl-β-cyclodextrin (MCD) was obtained from Sigma-Aldrich (St. Louis, Mo., USA). MK571 was obtained from EMD Chemicals (Philadelphia, Pa., USA).

### Cell Culture.

SKOV3 and CaOV3 cell lines were obtained from ATCC (Manassus, Va., USA). HEY cells were obtained from Dr. Gordon Mills (MD Anderson Cancer Center, Houston, Tex., USA) and were certified by Biosynthesis (Lewisville, Tex., USA). SKOV/VLB, a SKOV3-derived multidrug-resistant cell line expressing high levels of P-glycoprotein (17) was obtained from Dr. Jian-ting Zhang, IUSM, and was certified by Biosynthesis (Lewisville, Tex., USA). SKOV3-Luc cells (a gift from Dr. Melissa Fishel, IUSM), which express both green fluorescent protein and luciferase, were used for the mouse models of EOC. EOC cells were cultured in RPMI with 5% FBS and antibiotics. RAW 264.7 mouse macrophages were obtained from ATCC and cultured in DMEM with 10% FBS and antibiotics. All cell lines were maintained in a humidified incubator at 37° C. and 5% CO₂ and were used within 20 passages of receipt or certification.

### ATX Enzymatic Activity Analyses.

Tissue homogenate (0.02 mg protein) or human EOC ascites (1.0 μL) per assay were mixed with FS-3 (0.5 μg in 200 μL of PBS) in a 96-well plate and incubated at 37° C. The fluorescence was read at intervals over several hours on a Victor³V plate reader (Perkin Elmer, Waltham, Mass., USA). ATX activity was expressed as change in fluorescence intensity/min/mg of protein or /μL of body fluid.

### Proliferation, Migration/Invasion and Lipid Analyses.

Proliferation assays using MTT, cell migration and invasion assays were conducted as described previously (2). Proliferation data are presented as mean and SD of the OD 555 for 4-6 wells. Migration/invasion data are presented as mean and SD of cells/field/membrane for at least 3 membranes. Lipids were extracted using a LPL extraction method (the MeOH method) (18) and mass spectrometry analyses were performed using API-4000 (Applied Biosystems/MDS SCIEX, Carlsbad, Calif.) with the Analyst data acquisition system as described previously.

### Quantitative Real Time PCR.

SKOV3 cells in 6-well plate were incubated with 33 nM control siRNA or one of the LPA₁₋₄ siRNAs for 60 hours. Then SKOV3 cells were collected in the QIAGEN RLT lysis buffer. RNA was extracted with an RNeasy mini kit (QIAGEN) and reverse transcribed by M-MLV reverse transcriptase. Quantitative real-time PCR was performed on Light Cycler 480 (Roche). Primer sequences are: GAPDH-F: 5′-GAAGGTGAAGGTCGGAGT-3′ (SEQ ID NO. 1); GAPDH-R: 5′-GAAGATGGTGATGGGATTTC-3′(SEQ ID NO. 2); LPA₁-F: 5′-AATCGAGAGGCACATTACGG-3′ (SEQ ID NO. 3); LPA₁-R: 5′-GTTGAAAATGGCCCAGAAGA-3′ (SEQ ID NO. 4); LPA₂-F: 5′-TTGTCTTCCTGCTCATGGTG-3′ (SEQ ID NO. 5); LPA₂-R: 5′-TCAGCATCTCGGCAAGAGTA-3′ (SEQ ID NO. 6); LPA₃-F: 5′-TGCTCATTTTGCTTGTCTGG-3′ (SEQ ID NO. 7); LPA₃-R: 5′-GCCATACATGTCCTCGTCCT-3′ (SEQ ID NO. 8); LPA₄-F: 5′-CTTCGCAAGCCTGCTACTCT-3′(SEQ ID NO. 9); and LPA₄-R: 5′-GGCTTTGTGGTCAAAGGTGT-3′(SEQ ID NO. 10).

### Mouse Xenograft Model of EOC.

Female NOD/SCID mice were obtained from the In Vivo Therapeutics Core, IUSM (Indianapolis, Ind.) at 6 to 8 wks of age. All animal protocols were approved by the IUSM Animal Care and Use Committee. The xenograft models used were essentially the same as the ones described previously (2). In the first study, SKOV3-Luc cells (10⁷ in 500 μL, PBS) were injected i.p. into mice (n=12 for each group) on day O, Starting at day 10, the mice were injected i.p. with MAFP (0.22 mg/kg.) or vehicle three times per wk for 4 wks. Thirty-eight to 40 days after tumor cell injections, mice were sacrificed and tumor development was assessed. Tumors were counted at each metastatic location, and tumor diameters and volume of ascites were measured. For testing the potential toxic effects on tissues, fixed paraffin-imbedded tissue slices from the kidney, liver, small intestine, lung, and brain were subjected to H&E staining and pathologic examination. For the second study, NOD/SCID mice (n=7) were injected i.p. with 10⁶ HEY cells in 500 μL PBS. After two days, 0.5 mL S4 ascites (mixture of 3 specimens) or 0.5 mL PBS was injected i.p. into the mice, and this was continued 3 times/wk for 3 wks. Mice were sacrificed and tumor and ascites development was assessed as described above. In a third study, 10⁷ SKOV3-Luc cells were injected i.p. into NOD/SCID mice (n=6). After 10 days, a pooled P3 fraction (containing 500 μg protein) was injected i.p. and this was continued 2 times/wk for 3 wks. Mice were sacrificed and tumor and ascites development were assessed.

### Statistical Analyses.

Data are presented as mean±SD. For differences between two groups the Students t-test was employed. For differences between multiple groups or treatments, one-way ANOVA was performed with Dunnett's test to compare the treatments to the control. The statistical program GraphPad Prism 5, Ver. 5.04 (GraphPad Software, Inc., La Jolla, Calif.) was used for all analyses. The significance level was set at α=0.05.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

### REFERENCES

1. Sengupta S, Wang Z, Tipps R, Xu Y. Biology of LPA in health and disease. Semin Cell Dev Biol. 2004 October; 15(5):503-12.
2. Xu Y, Sengupta S, Singh S, Steinmetz R. Novel lipid signaling pathways in ovarian cancer cells. Cell Science Reviews 2006; 3:168-97.
3. Mills G B, Moolenaar W H. The emerging role of lysophosphatidic acid in cancer. Nat Rev Cancer. 2003 August; 3(8):582-91.
4. Xu Y, Casey G, Mills G B. Effect of lysophospholipids on signaling in the human Jurkat T cell line. J Cell Physiol. 1995 June; 163(3):441-50.
5. Xu Y, Fang X J, Casey G, Mills G B. Lysophospholipids activate ovarian and breast cancer cells. Biochem J. 1995 Aug. 1; 309 (Pt 3):933-40.
6. Shen Z, Belinson J, Morton R E, Xu Y, Xu Y. Phorbol 12-myristate 13-acetate stimulates lysophosphatidic acid secretion from ovarian and cervical cancer cells but not from breast or leukemia cells. Gynecol Oncol. 1998 December; 71(3):364-8.
7. Xu Y, Shen Z, Wiper D W, Wu M, Morton R E, Elson P, et al. Lysophosphatidic acid as a potential biomarker for ovarian and other gynecologic cancers. Jama. 1998 Aug. 26; 280(8):719-23.
8. Xiao Y, Chen Y, Kennedy A W, Belinson J, Xu Y. Evaluation of plasma lysophospholipids for diagnostic significance using electrospray ionization mass spectrometry (ESI-MS) analyses. Ann N Y Acad. Sci. 2000 April; 905:242-59.
9. Schwartz B M, Hong G, Morrison B H, Wu W, Baudhuin L M, Xiao Y J, et al. Lysophospholipids increase interleukin-8 expression in ovarian cancer cells. Gynecol Oncol. 2001 May; 81(2):291-300.
10. Shen Z, Wu M, Elson P, Kennedy A W, Belinson J, Casey G, et al. Fatty acid composition of lysophosphatidic acid and lysophosphatidylinositol in plasma from patients with ovarian cancer and other gynecological diseases. Gynecol Oncol. 2001 October; 83(1):25-30.
11. Xiao Y J, Schwartz B, Washington M, Kennedy A, Webster K, Belinson J, et al. Electrospray ionization mass spectrometry analysis of lysophospholipids in human ascitic fluids: comparison of the lysophospholipid contents in malignant vs nonmalignant ascitic fluids. Anal Biochem. 2001 March; 290(2):302-13.
12. Xu Y, Xiao Y J, Baudhuin L M, Schwartz B M. The role and clinical applications of bioactive lysolipids in ovarian cancer. J Soc Gynecol Investig. 2001 January-February; 8(1):1-13.
13. Baudhuin L M, Cristina K L, Lu J, Xu Y. Akt activation induced by lysophosphatidic acid and sphingosine-1-phosphate requires both mitogen-activated protein kinase kinase and p38 mitogen-activated protein kinase and is cell-line specific. Mol. Pharmacol. 2002 September; 62(3):660-71.
14. Sengupta S, Xiao Y J, Xu Y. A novel laminin-induced LPA autocrine loop in the migration of ovarian cancer cells. Faseb J. 2003 August; 17(11):1570-2.
15. Xu Y, Xiao Y J, Zhu K, Baudhuin L M, Lu J, Hong G, et al. Unfolding the pathophysiological role of bioactive lysophospholipids. Curr Drug Targets Immune Endocr Metabol Disord. 2003 March; 3(1):23-32.
16. Baudhuin L M, Jiang Y, Zaslaysky A, Ishii I, Chun J, Xu Y. S1P3-mediated Akt activation and cross-talk with platelet-derived growth factor receptor (PDGFR). Faseb J. 2004 February; 18(2):341-3.
17. Sutphen R, Xu Y, Wilbanks G D, Fiorica J, Grendys E C, Jr., LaPolla J P, et al. Lysophospholipids are potential biomarkers of ovarian cancer. Cancer Epidemiol Biomarkers Prey. 2004 July; 13 (7): 1185-91.
18. Kim K S, Ren J, Jiang Y, Ebrahem Q, Tipps R, Cristina K, et al. GPR4 plays a critical role in endothelial cell function and mediates the effects of sphingosylphosphorylcholine. Faseb J. 2005 May; 19(7):819-21.
19. Wang P, Wu X H, Chen W X, Shan B E, Guo Q. [Expression of lysophosphatidic acid receptor in human ovarian cancer cell lines 3AO, SKOV3, OVCAR3 and its significance]. Di Yi Jun Yi Da Xue Xue Bao. 2005 November; 25(11):1422-4, 31.
20. Kim K S, Sengupta S, Berk M, Kwak Y G, Escobar P F, Belinson J, et al. Hypoxia enhances lysophosphatidic acid responsiveness in ovarian cancer cells and lysophosphatidic acid induces ovarian tumor metastasis in vivo. Cancer Res. 2006 Aug. 15; 66(16):7983-90.
21. Ren J, Xiao Y J, Singh L S, Zhao X, Zhao Z, Feng L, et al. Lysophosphatidic acid is constitutively produced by human peritoneal mesothelial cells and enhances adhesion, migration, and invasion of ovarian cancer cells. Cancer Res. 2006 Mar. 15; 66(6):3006 14.
22. Zhao X, Wang D, Zhao Z, Xiao Y, Sengupta S, Xiao Y, et al. Caspase-3-dependent activation of calcium-independent phospholipase A2 enhances cell migration in non-apoptotic ovarian cancer cells. J Biol. Chem. 2006 Sep. 29; 281(39):29357-68.
23. Sengupta S, Kim K S, Berk M P, Oates R, Escobar P, Belinson J, et al. Lysophosphatidic acid downregulates tissue inhibitor of metalloproteinases, which are negatively involved in lysophosphatidic acid-induced cell invasion. Oncogene. 2007 May 3; 26(20):2894-901.
24. Wang H, Wu X H, Shi L. [Lysophosphatidic acid promotes transplanted tumor growth of human epithelium ovarian cancer in nude mice]. Nan Fang Yi Ke Da Xue Xue Bao. 2007 February; 27(2):228-9, 31.
25. Li H, Wang D, Zhang H, Kirmani K, Zhao Z, Steinmetz R, et al. Lysophosphatidic acid stimulates cell migration, invasion, and colony formation as well as tumorigenesis/metastasis of mouse ovarian cancer in immunocompetent mice. Mol Cancer Ther. 2009 June; 8(6):1692-701.
26. Xu Y, Wang D, Wang Z. Lipid generation and signaling in ovarian cancer. In: Stack M S, Fishman D A, editors. Cancer Treatment and Research-Ovarian cancer. 2 ed. New York, Dordrecht, Heidelberg, London: Springer; 2009. p. 241-68.
27. Murph M M, Liu W, Yu S, Lu Y, Hall H, Hennessy B T, et al. Lysophosphatidic acid-induced transcriptional profile represents serous epithelial ovarian carcinoma and worsened prognosis. PLoS ONE. 2009; 4(5):e5583.
28. Aoki J, Inoue A, Okudaira S. Two pathways for lysophosphatidic acid production. Biochim Biophys Acta. 2008 September; 1781(9):513-8.
29. Balsinde J, Balboa M A, Insel P A, Dennis E A. Regulation and inhibition of phospholipase A2. Annu Rev Pharmacol Toxicol. 1999; 39:175-89.
30. Kudo I, Murakami M. [Phospholipase A2]. Tanpakushitsu Kakusan Koso. 1999 June; 44(8 Suppl): 1013-24.
31. Kudo I, Murakami M. Phospholipase A2 enzymes. Prostaglandins Other Lipid Mediat. 2002 August; 68-69:3-58.
32. Aoki J. Mechanisms of lysophosphatidic acid production. Semin Cell Dev Biol. 2004 October; 15(5):477-89.
33. Tang J, Kriz R W, Wolfman N, Shaffer M, Seehra J, Jones S S. A novel cytosolic calcium-independent phospholipase A2 contains eight ankyrin motifs. J Biol. Chem. 1997 Mar. 28; 272(13):8567-75.
34. Ramanadham S, Yarasheski K E, Silva M J, Wohltmann M, Novack D V, Christiansen B, et al. Age-related changes in bone morphology are accelerated in group VIA phospholipase A2 (iPLA₂beta)-null mice. Am J. Pathol. 2008 April; 172(4):868-81.
35. Song Y, Wilkins P, Hu W, Murthy K S, Chen J, Lee Z, et al. Inhibition of calcium-independent phospholipase A2 suppresses proliferation and tumorigenicity of ovarian carcinoma cells. Biochem J. 2007 Sep. 15; 406(3):427-36.
36. Sedlakova I, Vavrova J, Tosner J, Hanousek L. Lysophosphatidic acid: an ovarian cancer marker. Eur J Gynaecol Oncol. 2008; 29(5):511-4.
37. Sedlakova I, Vavrova J, Tosner J, Hanousek L. [Lysophosphatidic acid in ovarian cancer patients]. Ceska Gynekol. 2006 July; 71(4):312-7.
38. Seballos L, Zhang J Z, Sutphen R. Surface-enhanced Raman scattering detection of lysophosphatidic acid. Anal Bioanal Chem. 2005 November; 383(5):763-7.
39. Meleh M, Pozlep B, Mlakar A, Meden-Vrtovec H, Zupancic-Kralj L. Determination of serum lysophosphatidic acid as a potential biomarker for ovarian cancer. J Chromatogr B Analyt Technol Biomed Life Sci. 2007 Oct. 15; 858(1-2):287-91.
40. Jeon E S, Moon H J, Lee M J, Song H Y, Kim Y M, Cho M, et al. Cancer-derived lysophosphatidic acid stimulates differentiation of human mesenchymal stem cells to myofibroblast-like cells. Stem Cells. 2008 March; 26(3):789-97.
41. Kosaka N, Iguchi H, Ochiya T. Circulating microRNA in body fluid: a new potential biomarker for cancer diagnosis and prognosis. Cancer Sci. 2010 Jun. 18.
42. Taylor D D, Gercel-Taylor C. MicroRNA signatures of tumor-derived exosomes as diagnostic biomarkers of ovarian cancer. Gynecol Oncol. 2008 July; 110(1):13-21.
43. Subra C, Grand D, Laulagnier K, Stella A, Lambeau G, Paillasse M, et al. Exosomes account for vesicle-mediated transcellular transport of activatable phospholipases and prostaglandins. J Lipid Res. 2010 August; 51(8):2105-20.
44. Burke J E, Dennis E A. Phospholipase A2 structure/function, mechanism, and signaling. J Lipid Res. 2009 April; 50 Suppl:S237-42.
45. Hazen S L, Zupan L A, Weiss R H, Getman D P, Gross R W. Suicide inhibition of canine myocardial cytosolic calcium-independent phospholipase A2. Mechanism-based discrimination between calcium-dependent and -independent phospholipases A2. J Biol. Chem. 1991 Apr. 15; 266(11):7227-32.
46. Burke J E, Dennis E A. Phospholipase A2 biochemistry. Cardiovasc Drugs Ther. 2009 February; 23(1):49-59.
47. Baskakis C, Magrioti V, Cotton N, Stephens D, Constantinou-Kokotou V, Dennis E A, et al. Synthesis of polyfluoro ketones for selective inhibition of human phospholipase A2 enzymes. J Med. Chem. 2008 Dec. 25; 51(24):8027-37.
48. Lopez-Vales R, Navarro X, Shimizu T, Baskakis C, Kokotos G, Constantinou-Kokotou V, et al. Intracellular phospholipase A(2) group IVA and group VIA play important roles in Wallerian degeneration and axon regeneration after peripheral nerve injury. Brain. 2008 October; 131(Pt 10):2620-31.
49. Cummings B S. Phospholipase A2 as targets for anti-cancer drugs. Biochem Pharmacol. 2007 Oct. 1; 74(7):949-59.
50. Gorovetz M, Baekelandt M, Berner A, Trope C G, Davidson B, Reich R. The clinical role of phospholipase A2 isoforms in advanced-stage ovarian carcinoma. Gynecol Oncol. 2006 December; 103(3):831-40.
51. Iorio E, Ricci A, Bagnoli M, Pisanu M E, Castellano G, Di Vito M, et al. Activation of phosphatidylcholine cycle enzymes in human epithelial ovarian cancer cells. Cancer Res. 2010 Mar. 1; 70(5):2126-35.
52. Li H, Zhao Z, Wei G, Yan L, Wang D, Zhang H, et al. Group VIA phospholipase A2 in both host and tumor cells is involved in ovarian cancer development. FASEB J. 2010 Jun. 8.
53. van't Veer L J, Dai H, van de Vijver M J, He Y D, Hart A A, Mao M, et al. Gene expression profiling predicts clinical outcome of breast cancer. Nature. 2002 Jan. 31; 415(6871):530-6.
54. van de Vijver M J, He Y D, van't Veer L J, Dai H, Hart A A, Voskuil D W, et al. A gene-expression signature as a predictor of survival in breast cancer. N Engl J. Med. 2002 Dec. 19; 347(25):1999-2009.
55. Zhao Z, Xiao Y, Elson P, Tan H, Plummer S J, Berk M, et al. Plasma lysophosphatidylcholine levels: potential biomarkers for colorectal cancer. J Clin Oncol. 2007 Jul. 1; 25(19):2696-701.
56. Zhao Z, Xu Y. Measurement of endogenous lysophosphatidic acid by ESI-MS/MS in plasma samples requires pre-separation of lysophosphatidylcholine. J Chromatogr B Analyt Technol Biomed Life Sci. 2009 Nov. 1; 877(29):3739-42.
57. Zhao Z, Xu Y. An extremely simple method for extraction of lysophospholipids and phospholipids from blood samples. J Lipid Res. 2009 Sep. 25.
58. Li H, Wang D, Singh L S, Berk M, Tan H, Zhao Z, et al. Abnormalities in osteoclastogenesis and decreased tumorigenesis in mice deficient for ovarian cancer G protein-coupled receptor 1. PLoS One. 2009; 4(5):e5705.
59. Vang R, Shih Ie M, Kurman R J. Ovarian low-grade and high-grade serous carcinoma: pathogenesis, clinicopathologic and molecular biologic features, and diagnostic problems. Adv Anat Pathol. 2009 September; 16(5):267-82.
60. Kurman R J, Shih Ie M. Pathogenesis of ovarian cancer: lessons from morphology and molecular biology and their clinical implications. Int J Gynecol Pathol. 2008 April; 27(2): 151-60.
61. Bast R C, Jr., Hennessy B, Mills G B. The biology of ovarian cancer: new opportunities for translation. Nat Rev Cancer. 2009 June; 9(6):415-28.
62. Schlaerth A C, Abu-Rustum N R. Role of minimally invasive surgery in gynecologic cancers. Oncologist. 2006 September; 11(8):895-901.
63. Shao M, Cao L, Shen C, Satpathy M, Chelladurai B, Bigsby R M, et al. Epithelial-to-mesenchymal transition and ovarian tumor progression induced by tissue transglutaminase. Cancer Res. 2009 Dec. 15; 69(24):9192-201.
64. Jiang Y, Berk M, Singh L S, Tan H, Yin L, Powell C T, et al. KiSS1 suppresses metastasis in human ovarian cancer via inhibition of protein kinase C alpha. Clin Exp Metastasis. 2005; 22(5):369-76.
65. Yang G, Rosen D G, Mercado-Uribe I, Colacino J A, Mills G B, Bast R C, Jr., et al. Knockdown of p53 combined with expression of the catalytic subunit of telomerase is sufficient to immortalize primary human ovarian surface epithelial cells. Carcinogenesis. 2007 January; 28(1):174-82.
66. Wang D, Zhao Z, Caperell-Grant A, Yang G, Mok S C, Liu J, et al. S11³ differentially regulates migration of human ovarian cancer and human ovarian surface epithelial cells. Mol Cancer Ther. 2008 July; 7(7):1993-2002.
67. Murakami M, Kudo I. Phospholipase A2. J. Biochem. 2002 March; 131(3):285-92.
68. Lauber K, Bohn E, Krober S M, Xiao Y J, Blumenthal S G, Lindemann R K, et al. Apoptotic cells induce migration of phagocytes via caspase-3-mediated release of a lipid attraction signal. Cell. 2003 Jun. 13; 113(6):717-30.
69. Flesken-Nikitin A, Choi K C, Eng J P, Shmidt E N, Nikitin A Y. Induction of carcinogenesis by concurrent inactivation of p53 and Rb1 in the mouse ovarian surface epithelium. Cancer Res. 2003 Jul. 1; 63(13):3459-63.
70. Dinulescu D M, Ince T A, Quade B J, Shafer S A, Crowley D, Jacks T. Role of K-ras and Pten in the development of mouse models of endometriosis and endometrioid ovarian cancer. Nat. Med. 2005 January; 11(1):63-70.
71. Vanderhyden B C, Shaw T J, Ethier J F. Animal models of ovarian cancer. Reprod Biol Endocrinol. 2003 Oct. 7; 1:67.
72. Fong M Y, Kakar S S. Ovarian cancer mouse models: a summary of current models and their limitations. J Ovarian Res. 2009; 2(1):12.
73. Ackermann E J, Kempner E S, Dennis E A. Ca(2+)-independent cytosolic phospholipase A2 from macrophage-like P388D1 cells. Isolation and characterization. J Biol. Chem. 1994; 269(12):9227-33.
74. Deems R A, Eaton B R, Dennis E A. Kinetic analysis of phospholipase A2 activity toward mixed micelles and its implications for the study of lipolytic enzymes. J Biol. Chem. 1975; 250(23):9013-20.
75. Lucas K K, Dennis E A. Distinguishing phospholipase A2 types in biological samples by employing group-specific assays in the presence of inhibitors. Prostaglandins Other Lipid Mediat. 2005; 77(1-4):235-48.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

What is claimed is:

1. A diagnostic method, comprising the steps of:
measuring PLA₂ activity in a sample from a patient; and
assigning said patient to a group, wherein the group includes patients having a diagnosis of EOC, if the level of PLA₂ activity in said sample is greater than or equal to about three times the level of PLA₂ activity in a sample from an individual that does not have a diagnosis EOC.

2. The method according to claim 1, further including the step of:
obtaining said patient sample from a patient.

3. The method according to claim 1, wherein said patient sample is human ascites.

4. The method according to claim 1, wherein said patient sample is human blood.

5. The method according to claim 1, wherein the sample has levels of cell free and vesicle free cPLA₂ and iPLA₂ in the range of about 0.5 times higher than is found in a similar sample from an individual that does not have a diagnosis of EOC.

6. The method according to claim 1, wherein the measuring step includes the use of a fluorogenic compound.

7. The method according to claim 6, wherein the fluorogenic compound is DBPC.

8. The method according to claim 1, wherein the measuring step includes the use a radioactive labeled substrate for PLA₂.

9. The method according to claim 1, further including the step of separating the soluble, cell free and vesicle free fraction of the sample from the total sample and measuring PLA₂ in the soluble vesicle free fraction.

10. The method according to claim 1, further including the step of:
contacting a portion of the cell free and vesicle free sample with at least one compound, wherein the compound inhibits the activity of at least one isoform of PLA₂.

11. The method according to claim 10, wherein the compound inhibits the activity of sPLA₂.

12. A method of screening for compounds to treat EOC, comprising the steps of:
contacting a compound with at least one isoforms of PLA₂, wherein the isoform of PLA₂.selected from the group consisting of: cPLA₂ and iPLA₂; and
measuring the effect of the compound on the activity one at least one of said isoforms of PLA₂.

13. The method according to claim 12, further including the step of:
adding a compound to an assay that includes ATX; and
determining if the compound effects the activity of ATX.

14. The method according to claim 12, further including the step of:
introducing a EOC cancer cell to an assay that includes an active form of at least one of the enzymes selected from the group consisting of cPLA₂, iPLA₂, and ATX, wherein the assay include a portion of serum free growth media conducive to eukaryotic cell growth and reproduction;
supplying the at least one compound; and
assaying to determine if the compound effects the growth or reproduction of the EOC cancer cell and/or the ability of cells to migrate.

15. The method according to claim, 14, wherein the EOC cancer cell is an SKOV3 cell.

16. The method according to claim, 14, wherein the EOC cancer cell is an HEY cell.

17. The method according to claim 14, wherein the assay further includes the cell free and vesicle free fraction recovered from an EOC ascites.

18. A method of treating EOC, comprising the steps of:
administering a therapeutically effective of a compound that inhibits an isoform of PLA₂ to a patient in need thereof.

19. The method according to claim 18, wherein the isoform of PLA₂, is selected from the group consisting of cPLA₂, and iPLA₂.

20. The method according to claim 18, wherein the patient is a human being.
